SEQUENCE LISTING (23 sequences)

SEQ ID NO: 1 - DNA, 1419 bp, Sphingomonas subterranea
  CDS (1)..(1416)
atggcaagtgcagcagcgggtgcagacggccttcccttgctcgatggcggcgtgccgctgctcgggcacctcgcccagttctttcgcgatccggtttccgtcctgaagcgcggctatcgctcgaagggccggctcttcgcgatgaacttcatgggccagcgcatgaacgtgatgctggggccggaacacaaccgcttcttcttcgaggaaaccgacaagctgctctcgatccgagagtcgatgccgttcttcctcaagatgttctcgcccgagttctactcgtttgccgagatggacgaatacctgcgccagcgcgcgatcatcatgccccggttcaaggcggcatcgatgaagcagtacgtgccggtgatggtcgaggaatcgctgaacctcgtcgaacggctgggtgaggaaggcgagttcgacctgatcccgacgctgggccccgtggtaatggacatcgccgcgcacagcttcatggggcgcgaattccacgagaagcttggccacgaattcttcgagctgttccgcgacttctcgggcggcatggagttcgtgctgccgctgtggctgccgacaccaaagatggtgaaaagccagcgcgccaagaagaagctccacgcgatcctgcaatcgtggatcgacaagcgccgcgccagtccgctcgatccgcccgacttcttccagacgatgatcgagacgaagtatcccgatggccgcgccgtgcccgacgagatcatccggcacctgatcctgctgctggtctgggcgggccacgaaaccaccgccgggcaagtcagctgggcgctggccgacctcctccagaaccccggctaccagcaggtgctgcgcgaagaggtcgcctcgctgctcggcggcagcgacgggcgcgcactgggctgggagcaggccgtggcgatggagaagatggacctcgccctgcgcgagacagagcggctccaccccgtcgcctacatgctcagccgcaaggccagtgccgatatcgagcgcgacggctatcgcatccgcaagggcgaattcgtgctgctcgcgccctcggtcagccatcgcatggaagaaaccttccgcaatgccgatgcctatgaccccgagcgcttcaacccgcagaacccggacgcgcagatcgagagcaactcgctgatcggcttcggcggcggggtgcatcgctgtgcgggggtaaacttcgcgcgcatggaaatgaaggtgctggtggcgatcctgctgcagaacttcgacatggagctgatcgacgaagtgcgccccatcgccggcgcatcgacctactggcccgcccagccctgccgcgtgcgctacaagcggcgcaagctggatggcgcgggtggaagcgcggacgtggcggcgctcgccaaggcggccggttgcccggcccatgcatga

SEQ ID NO: 2 - protein, 472 aa, Sphingomonas subterranea
MASAAAGADGLPLLDGGVPLLGHLAQFFRDPVSVLKRGYRSKGRLFAMNFMGQRMNVMLGPEHNRFFFEETDKLLSIRESMPFFLKMFSPEFYSFAEMDEYLRQRAIIMPRFKAASMKQYVPVMVEESLNLVERLGEEGEFDLIPTLGPVVMDIAAHSFMGREFHEKLGHEFFELFRDFSGGMEFVLPLWLPTPKMVKSQRAKKKLHAILQSWIDKRRASPLDPPDFFQTMIETKYPDGRAVPDEIIRHLILLLVWAGHETTAGQVSWALADLLQNPGYQQVLREEVASLLGGSDGRALGWEQAVAMEKMDLALRETERLHPVAYMLSRKASADIERDGYRIRKGEFVLLAPSVSHRMEETFRNADAYDPERFNPQNPDAQIESNSLIGFGGGVHRCAGVNFARMEMKVLVAILLQNFDMELIDEVRPIAGASTYWPAQPCRVRYKRRKLDGAGGSADVAALAKAAGCPAHA

SEQ ID NO: 3 - DNA, 1762 bp, Sphingomonas subterranea
atggcaagtgcagcagcgggtgcagacggccttcccttgctcgatggcggcgtgccgctgctcgggcacctcgcccagttctttcgcgatccggtttccgtcctgaagcgcggctatcgctcgaagggccggctcttcgcgatgaacttcatgggccagcgcatgaacgtgatgctggggccggaacacaaccgcttcttcttcgaggaaaccgacaagctgctctcgatccgagagtcgatgccgttcttcctcaagatgttctcgcccgagttctactcgtttgccgagatggacgaatacctgcgccagcgcgcgatcatcatgccccggttcaaggcggcatcgatgaagcagtacgtgccggtgatggtcgaggaatcgctgaacctcgtcgaacggctgggtgaggaaggcgagttcgacctgatcccgacgctgggccccgtggtaatggacatcgccgcgcacagcttcatggggcgcgaattccacgagaagcttggccacgaattcttcgagctgttccgcgacttctcgggcggcatggagttcgtgctgccgctgtggctgccgacaccaaagatggtgaaaagccagcgcgccaagaagaagctccacgcgatcctgcaatcgtggatcgacaagcgccgcgccagtccgctcgatccgcccgacttcttccagacgatgatcgagacgaagtatcccgatggccgcgccgtgcccgacgagatcatccggcacctgatcctgctgctggtctgggcgggccacgaaaccaccgccgggcaagtcagctgggcgctggccgacctcctccagaaccccggctaccagcaggtgctgcgcgaagaggtcgcctcgctgctcggcggcagcgacgggcgcgcactgggctgggagcaggccgtggcgatggagaagatggacctcgccctgcgcgagacagagcggctccaccccgtcgcctacatgctcagccgcaaggccagtgccgatatcgagcgcgacggctatcgcatccgcaagggcgaattcgtgctgctcgcgccctcggtcagccatcgcatggaagaaaccttccgcaatgccgatgcctatgaccccgagcgcttcaacccgcagaacccggacgcgcagatcgagagcaactcgctgatcggcttcggcggcggggtgcatcgctgtgcgggggtaaacttcgcgcgcatggaaatgaaggtgctggtggcgatcctgctgcagaacttcgacatggagctgatcgacgaagtgcgccccatcgccggcgcatcgacctactggcccgcccagccctgccgcgtgcgctacaagcggcgcaagctggatggcgcgggtggaagcgcggacgtggcggcgctcgccaaggcggccggttgcccggcccatgcatgaggcgcactgaatggccaaggtgactttcgtccagccggacggctcggcgcggacttgcgtgaacttcgagggcatgaccctgatgcagctcgcggtcggcaaccttgtcgacgggatcgacgcgctgtgcggcggcatgatgcagtgcgccacctgccattgctacatcgacccggactggcttgaccgcaccggccccgcccggccggaagaacgcgagatgctcgaggccatcgatggcgtcgagatccgcccgaacagccgcctgtcctgccaggtccagcttggcgaggagttggacgggctggtggtgcacattccggcggagcaaccgggagtttag

SEQ ID NO: 4 - DNA, 1765 bp, Novosphingobium aromaticivorans
atggcaagagctgcgactgcggccggtaatggccttcccttgctcgatggaggcgtgccgctcctcgggcatctcgcacagttcttccgcgatccggtttcggtactcaagcgcggataccgctcgaaggggcggctcttcgcgatgaacttcatgggccagcgcatgaacgtgatgctgggtccggaacacaaccgcttcttcttcgaggagacggacaagctgctctcgatccgggagtcgatgccgttcttcctcaagatgttctcgcccgagttctattcgttcgcggaaatggacgagtacctgcgccagcgctcgatcatcatgccccgcttcaaggcggcatcgatgaagcagtacgtgccggtcatggtcgaggaatcgcttaacctggtcgagcggctgggcgaggaaggcgagttcgacctgatcccgacgctgggcccggtggtaatggacatcgccgcgcacagcttcatgggacgcgagttccacgagaagctggggcatgagttcttcgaactcttccgcgatttttcgggaggcatggaattcgtcctgccgctgtggctgccgacacccaagatggtcaagtcacagcgcgcgaagaggaagctccacgccatcctgcaatcgtggatcgacaagcgccgcgccgccccgctcgatccgcccgatttcttccagacgatgatcgagacgaagtatcccgatggccgcccggtgcccgacgagatcatccgccacctgatcctccttctcgtctgggcagggcacgagacgaccgccgggcaggtgagctgggcgctggcggacctccttcagaacccggactaccagaaggtgctgcgcggcgagatatcgtcgctgctgggcggcagcgacgggcgcgaccttggctgggaacaggccgtggcgatggagaagatggaccttgccctgcgcgagaccgagcggctccatccggtcgcctacatgctcagccgcaaggcgcgggccgatatcgagcgcgacggctatgtcatccgcaagggcgagttcgtgctgcttgcgccttcggtcagccaccgcatggaagagacgttccgcaatcccgatgcctatgacccggaacgcttcaacccggccaaccccgatgcgcagatcgaaagcaattcgttgatcggctttggcgggggtgtccaccgctgcgcgggcgtgaacttcgcgcggatggagatgaaggtgctggtggcgatcctgctccagaacttcgacatggagctgatggacgaagtgcggcccatcgcgggcgcatcgacctactggcccgcccagccctgccgggtgcgctatcggcggcgcaagctcgacgggtcggaggcaggtgcggacatggcggcgctggcccgagccgccggctgcccggcgcatacgtgagggaggcctgatggccaaggtgactttcgtccagccggacggatcgcagcgaacctgcgtgaacttcgaaggcatgacgttgatgcagctcgcagtgggcaatctcgtcgacgggatcgacgcgctgtgcggcggcatgatgcagtgcgcgacctgccattgctggatcgaccccgaatggatcggccgcaccggcatggccggacccgatgagcgggcaatgctggaagcgatcgagggcgtcgagatccgtcccgaaagccgcctgtcctgccaggtacagcttggcgaagaacttgacgggctggtcgtgcgcattccaccggagcaaccgggagtttag

SEQ ID NO: 5 - DNA, 32 bp, Artificial Sequence
  Description of Artificial Sequence Synthetic primer
cgagatctgatgaacttcatgggccagcgcat

SEQ ID NO: 6 - DNA, 32 bp, Artificial Sequence
  Description of Artificial Sequence Synthetic primer
cgagatctgcagcggtggacacccccgccaaa

SEQ ID NO: 7 - DNA, 35 bp, Artificial Sequence
  Description of Artificial Sequence Synthetic primer
ccccatatggcaagtgcagcagcgggtgcagacgg

SEQ ID NO: 8 - DNA, 29 bp, Artificial Sequence
  Description of Artificial Sequence Synthetic primer
gcactagtctaaactcccggttgctccgg

SEQ ID NO: 9 - DNA, 30 bp, Artificial Sequence
  Description of Artificial Sequence Synthetic primer
gcccatatggcaagagctgcgactgcggcc

SEQ ID NO: 10 - DNA, 29 bp, Artificial Sequence
  Description of Artificial Sequence Synthetic primer
gcactagtctaaactcccggttgctccgg

SEQ ID NO: 11 - DNA, 29 bp, Artificial Sequence
  Description of Artificial Sequence Synthetic primer
ccgctagcgcaagagctgcgactgcggcc

SEQ ID NO: 12 - DNA, 29 bp, Artificial Sequence
  Description of Artificial Sequence Synthetic primer
gcggatccctaaactcccggttgctccgg

SEQ ID NO: 13 - DNA, 30 bp, Artificial Sequence; identical to SEQ ID NO: 9
  Description of Artificial Sequence Synthetic primer

SEQ ID NO: 14 - DNA, 30 bp, Artificial Sequence
  Description of Artificial Sequence Synthetic primer
gccctcgagtcacgtatgcgccgggcagcc

SEQ ID NO: 15 - DNA, 30 bp, Artificial Sequence
  Description of Artificial Sequence Synthetic primer
gcccatatggcaagtgcagcagcgggtgca

SEQ ID NO: 16 - DNA, 30 bp, Artificial Sequence
  Description of Artificial Sequence Synthetic primer
gccctcgagtcatgcatgggccgggcaacc

SEQ ID NO: 17 - DNA, 30 bp, Artificial Sequence
  Description of Artificial Sequence Synthetic primer
gcccatatggcttcaacacgtagtcctcgc

SEQ ID NO: 18 - DNA, 30 bp, Artificial Sequence
  Description of Artificial Sequence Synthetic primer
gccctcgagtcactgctgggttgcttcctg

SEQ ID NO: 19 - DNA, 45 bp, Artificial Sequence
  Description of Artificial Sequence Synthetic primer
ccgcatatggcttcaacacgtagtcctcgccccttcaatgagatc

SEQ ID NO: 20 - DNA, 35 bp, Artificial Sequence
  Description of Artificial Sequence Synthetic primer
gcactagttcactgctgggttgcttcctggttaaa

SEQ ID NO: 21 - DNA, 45 bp, Artificial Sequence
  Description of Artificial Sequence Synthetic primer
gcactagtaaggaaacagaccatgtccacacaggagcagaccccc

SEQ ID NO: 22 - DNA, 29 bp, Artificial Sequence
  Description of Artificial Sequence Synthetic primer
cgtgatcaggcatcagacacggcatcagg

SEQ ID NO: 23 - protein, 473 aa, Novosphingobium aromaticivorans
MARAATAAGNGLPLLDGGVPLLGHLAQFFRDPVSVLKRGYRSKGRLFAMNFMGQRMNVMLGPEHNRFFFEETDKLLSIRESMPFFLKMFSPEFYSFAEMDEYLRQRSIIMPRFKAASMKQYVPVMVEESLNLVERLGEEGEFDLIPTLGPVVMDIAAHSFMGREFHEKLGHEFFELFRDFSGGMEFVLPLWLPTPKMVKSQRAKRKLHAILQSWIDKRRAAPLDPPDFFQTMIETKYPDGRPVPDEIIRHLILLLVWAGHETTAGQVSWALADLLQNPDYQKVLRGEISSLLGGSDGRDLGWEQAVAMEKMDLALRETERLHPVAYMLSRKARADIERDGYVIRKGEFVLLAPSVSHRMEETFRNPDAYDPERFNPANPDAQIESNSLIGFGGGVHRCAGVNFARMEMKVLVAILLQNFDMELMDEVRPIAGASTYWPAQPCRVRYRRRKLDGSEAGADMAALARAAGCPAHT

(19) United States
(12) Patent Application Publication
(10) Pub. No.: US 2012/0178124 A1
(43) Pub. Date: Jul. 12, 2012

(54) STEROL SIDE CHAIN-CLEAVING ENZYME PROTEIN AND USE THEREOF

(75) Inventors: Masahiro Yamagishi, Tokyo (JP); Takeshi Sakamoto, Kanagawa (JP); Makoto Ueda, Kanagawa (JP); Masashi Itoh, Shizuoka (JP); Yoshikazu Fujii, Shizuoka (JP); Hiroki Kabumoto, Shizuoka (JP); Akira Arisawa, Shizuoka (JP); Tadashi Fujii, Shizuoka (JP)

(73) Assignee: MITSUBISHI CHEMICAL CORPORATION, Tokyo (JP)

(21) Appl. No.: 13/143,607

(22) PCT Filed: Jan. 7, 2009
(86) PCT No.: PCT/JP2009/050095

§ 371 Date: Sep. 13, 2011

(51) Int. Cl. C12P 33/00; C12N 15/52; C12N 1/15; C12N 9/96; C12N 1/21; C12N 1/19; C12N 9/00; C12N 15/63

(52) U.S. Cl. 435/52; 435/183; 536/23.2; 435/320.1; 435/188; 435/252.33; 435/252.3; 435/252.31; 435/252.34; 435/252.32; 435/252.35; 435/254.2; 435/254.22; 435/254.23; 435/254.21; 435/254.11; 435/254.3; 435/254.7

(57) ABSTRACT

It is an object of the present invention to obtain highly active P450scc enzyme protein which is an important enzyme protein that catalyzes the first step of the biosynthesis of industrially useful steroid hormone. The present invention provides a sterol side chain cleavage enzyme protein having the following physicochemical properties:
(1) action: the enzyme acts on sterol represented by formula (I) as defined in the specification and cleaves the carbon-carbon bond between positions 20 and 22 of a sterol side chain portion by its activity of cleaving the bonds, so as to generate a compound represented by formula (II) as defined in the specification;
(2) substrate specificity: when microorganisms that produce the enzyme protein are allowed to react with an aqueous solution containing 100 μg/ml 4-cholesten-3-one or cholesterol at 28° C. for 5 hours, the conversion reaction rate from 4-cholesten-3-one to progesterone is 10% or more, and the conversion rate from cholesterol to pregnenolone is 10% or more;

### TECHNICAL FIELD

The present invention relates to: an enzyme protein that cleaves the bond between positions 20 and 22 of a sterol side chain to produce pregnenolone and the like that are industrially useful as medicaments or pharmaceutical intermediates; DNA encoding the enzyme protein; a transformant obtained by introducing the DNA into a vector; a method for producing pregnenolone and the like, and hydrocortisone and a derivative thereof, using the enzyme protein; etc.

### BACKGROUND ART

11β,17α,21-trihydroxy-4-pregnen-3,20-dione (hydrocortisone), and its precursor substances, pregnenolone, progesterone, and 7-dehydropregnenolone, are compounds that are industrially useful as medicaments and pharmaceutical intermediates. As a conventional method for producing pregnenolone, naturally-occurring sterol compounds such as cholesterol, diosgenin and stigmasterol are used as raw materials, and such pregnenolone is produced by a plurality of organic synthetic reactions (Non-Patent Document 1). However, these methods are problematic as industrial production methods, in that they include many steps and the yield of the product of interest is low. On the other hand, as a method for biochemically producing hydrocortisone, the sterol conversion method, which uses the enzyme (side chain cleavage cytochrome P450; hereinafter referred to as “P450scc” at times) having activity of cleaving the bond between positions 20 and 22 of the sterol side chain derived from an animal, has been disclosed (Non-Patent Document 2). However, such an animal-derived enzyme protein has low activity. In addition, the culture of cells to be used as host cells requires an expensive medium, and a proliferation rate is low. Thus, this production method has not yet been sufficiently established. Moreover, there has also been disclosed a method comprising introducing bovine-derived P450scc into the yeast and then converting cholesterol to pregnenolone in the presence of adrenodoxin and adrenodoxin reductase that constitute the electron transfer system. However, productivity is extremely low, and thus, productivity at an industrial level has not yet been obtained (Patent Document 1).
[Non-Patent Document 1] J. Org. Chem., 1979, 44, 1583
[Non-Patent Document 2] Proc. Natl. Acad. Sci. USA, 1988, 85, 1988
[Patent Document 1] Japanese Patent No. 2963711

### DISCLOSURE OF THE INVENTION

### Problems to be Solved by the Invention

It is an object to be achieved by the present invention to provide highly active P450scc that catalyzes the first step of the biosynthesis of industrially useful steroid hormone, and a method for simply producing pregnenolone and the like, and further, hydrocortisone, at low costs, using the aforementioned P450scc.

### Means for Solving the Problems

As a result of intensive studies directed towards achieving the aforementioned object, the present inventors have measured several hundreds of P450scc from microorganisms, in terms of the activity of cleaving the bond between positions 20 and 22 of a sterol side chain, and have discovered an enzyme protein having extremely high activity of cleaving the bond between positions 20 and 22 of a sterol compound side chain. The inventors have discovered for the first time that this protein has the aforementioned activity. Moreover, the inventors have screened for DNAs encoding the P450 proteins of the closely-related microorganisms, using, as an indicator, high homology at the nucleotide sequence level to the aforementioned microorganism-derived enzyme protein. As a result, they have discovered DNA having a novel nucleotide sequence. The present inventors have found that products of interests, such as pregnenolone and the like, can be produced in high concentrations by producing a transformant using DNA encoding the above-described microorganism-derived enzyme protein, and then allowing the enzyme protein-transformed cells, the transformant-treated products and/or the culture to act on a sterol compound used as a raw material. The present invention has been completed based on these findings.

Thus, the present invention provides the following invention.

<1> A protein described in any one of the following (a), (b) and (c):
(a) a protein consisting of the amino acid sequence shown in SEQ ID NO: 2;
(b) a protein consisting of an amino acid sequence comprising a deletion, substitution and/or addition of one or several amino acids with respect to the amino acid sequence shown in SEQ ID NO: 2, and having activity of cleaving the bond between positions 20 and 22 of a sterol side chain, wherein the maximum velocity (Vmax) used as a reaction rate parameter of the aforementioned activity is 50 mmol/min/mol or more; and
(c) a protein consisting of an amino acid sequence having homology of 95% or more to the amino acid sequence shown in SEQ ID NO: 2, and having activity of cleaving the bond between positions 20 and 22 of a sterol side chain, wherein the maximum velocity (Vmax) used as a reaction rate parameter of the aforementioned activity is 50 mmol/min/mol or more.
<2> A sterol side chain cleavage enzyme having the following physicochemical properties:
(1) action: the enzyme acts on sterol represented by formula (I) as shown below and cleaves the carbon-carbon bond between positions 20 and 22 of a sterol side chain portion by its activity of cleaving the bonds, so as to generate a compound represented by formula (II) as shown below;
(2) substrate specificity: when microorganisms that produce the enzyme are allowed to react with an aqueous solution containing 100 μg/ml 4-cholesten-3-one or cholesterol at 28° C. for 5 hours, the conversion reaction rate from 4-cholesten-3-one to progesterone is 10% or more, and the conversion rate from cholesterol to pregnenolone is 10% or more;
(3) optimum pH: 7.5 to 8.0;
(4) optimum temperature for action: 15° C. to 20° C.;
(5) thermostability: after preservation at 20° C. for 140 hours, 30% or more of enzyme activity is maintained; and
(6) molecular weight: the putative molecular weight is assumed to be 53 to 54 KDa based on the amino acid sequence, and it is measured to be 50 to 56 KDa by SDS electrophoresis,

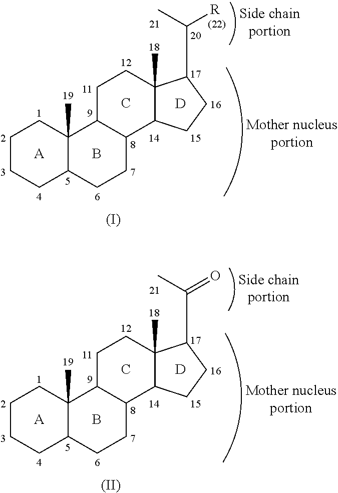

[wherein, in the formula (I),

the mother nucleus portion has a structure in which it consists of the A, B, C and D rings of a steroid, wherein

the mother nucleus portion has a carbon-carbon unsaturated bond(s) at zero site or at one or more sites of the positions 1 to 17 of the rings (except for the positions 10 and 13 thereof), and

carbon(s) at zero site or at one or more sites of the positions 1 to 19 of the rings (except for the positions 10 and 13 thereof) are independently substituted with

a group represented by the formula —OX

(namely, a hydroxy group wherein X═H; an acyloxyl group wherein X═COR₁ [wherein R₁ is a hydrogen atom, or an alkyl group, alkenyl group, alkynyl group or aromatic hydrocarbon containing 10 or less carbon atoms]; an O-alkyl group wherein X═R₂ [wherein R₂ is an alkyl group, alkenyl group or alkynyl group containing 10 or less carbon atoms, which may be optionally substituted with an oxygen atom]; a sulfate wherein X═SO₃M [wherein M is a hydrogen atom, an alkaline metal or an alkaline-earth metal]; an O-glycosyl group wherein X is the carbon at position 1 of a sugar; or an epoxy group wherein X is a carbon atom adjacent to said carbon), or

a keto group represented by the formula ═O, and

in the side chain portion, R is a linear alkyl group, alkenyl group or alkynyl group containing 10 or less carbon atoms, which may have a cyclic portion, or a branched alkyl group, alkenyl group or alkynyl group containing 10 or less carbon atoms, which may have a cyclic portion,

the carbons at positions 20 and 21, and at zero site or at one or more sites in the R, are independently substituted with

a group represented by the formula —OY

(namely, a hydroxy group wherein Y═H; an acyloxyl group wherein Y═COR₃ [wherein R₃ is a hydrogen atom, or an alkyl group, alkenyl group, alkynyl group or aromatic hydrocarbon containing 10 or less carbon atoms]; an O-alkyl group wherein Y═R₄ [wherein R₄ is an alkyl group, alkenyl group or allynyl group containing 10 or less carbon atoms, which may be optionally substituted with an oxygen atom]; a sulfate wherein Y═SO₃M [wherein M is a hydrogen atom, an alkaline metal or an alkaline-earth metal]; an O-glycosyl group wherein Y is the carbon at position 1 of a sugar; or an epoxy group wherein Y is a carbon atom adjacent to said carbon), or

a keto group represented by the formula ═O], and

[wherein, in the formula (II),

the mother nucleus portion has the same definitions as those of the mother nucleus portion of the formula (I), and

in the side chain portion, the carbon at position 21 is substituted with

zero or one group represented by the formula —OY

(namely, a hydroxy group wherein Y═H; an acyloxyl group wherein Y═COR₃ [wherein R₃ is a hydrogen atom, or an alkyl group, alkenyl group, alkynyl group or aromatic hydrocarbon containing 10 or less carbon atoms]; an O-alkyl group wherein Y═R₄ [wherein R₄ is an alkyl group, alkenyl group or alkynyl group containing 10 or less carbon atoms, which may be optionally substituted with an oxygen atom]; a sulfate wherein Y═SO₃M [wherein M is a hydrogen atom, an alkaline metal or an alkaline-earth metal]; or an O-glycosyl group wherein Y is the carbon at position 1 of a sugar), or

zero or one keto group represented by the formula ═O].

<3> DNA encoding the protein according to <1>.
<4> DNA described in any one of the following (a), (b) and (c):
(a) DNA having the nucleotide sequence shown in SEQ ID NO: 1;
(b) DNA having a nucleotide sequence comprising a deletion, substitution and/or addition of one or several nucleotides with respect to the nucleotide sequence shown in SEQ ID NO: 1, and encoding a protein which has activity of cleaving the bond between positions 20 and 22 of a sterol side chain, wherein the maximum velocity (Vmax) used as a reaction rate parameter of the aforementioned activity is 50 mmol/min/mol or more; and
(c) DNA having a nucleotide sequence capable of hybridizing under stringent conditions with DNA having the nucleotide sequence shown in SEQ ID NO: 1 or a complementary sequence thereof, and encoding a protein which has activity of cleaving the bond between positions 20 and 22 of a sterol side chain, wherein the maximum velocity (Vmax) used as a reaction rate parameter of the aforementioned activity is 50 mmol/min/mol or more.
<5> A fusion protein, wherein the enzyme protein according to <1> or <2>, a ferredoxin protein having electron-transferring activity on the enzyme protein, and a ferredoxin reductase protein having electron-transferring activity on the ferredoxin protein are allowed to bind to one another, so that they can function as a single protein.
<6> A recombinant vector comprising the DNA according to <3> or <4> and an expression control region capable of expressing a protein encoded by the DNA in a host cell.
<7> A transformant obtained by introducing the recombinant vector according to <6> into a host cell.
<8> A method for producing a compound represented by formula (II) as shown below, which comprises allowing a sterol represented by formula (I) as shown below to come into contact with the following (i) or (ii):
(i) a mixture of the enzyme protein according to <1> or <2>, a ferredoxin protein having electron-transferring activity on the enzyme protein, and a ferredoxin reductase protein having electron-transferring activity on the ferredoxin protein; or
(ii) the fusion protein according to <5>,

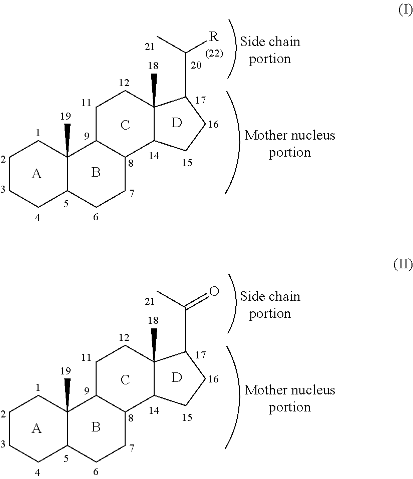

[wherein the definitions of the formula (I) and the formula (II) are the same as those of <1>].
<9> A method for producing a compound represented by formula (II) as shown below, which comprises allowing a sterol represented by formula (I) as shown below to come into contact with any one of the following (a) to (c):
(a) a mixture of a protein consisting of the amino acid sequence shown in SEQ ID NO: 23, a ferredoxin protein having electron-transferring activity on the aforementioned protein, and a ferredoxin reductase protein that transfers electron to the ferredoxin protein;
(b) a mixture of: a protein consisting of an amino acid sequence comprising a deletion, substitution and/or addition of one or several amino acids with respect to the amino acid sequence shown in SEQ ID NO: 23, and having activity of cleaving the bond between positions 20 and 22 of a sterol side chain, wherein the maximum velocity (Vmax) used as a reaction rate parameter of the aforementioned activity is 40 mmol/min/mol or more; a ferredoxin protein having electron-transferring activity on the aforementioned protein; and a ferredoxin reductase protein that transfers electron to the ferredoxin protein; and
(c) a fusion protein, wherein a protein consisting of the amino acid sequence shown in SEQ ID NO: 23 or a protein consisting of an amino acid sequence comprising a deletion, substitution and/or addition of one or several amino acids with respect to the amino acid sequence shown in SEQ ID NO: 23, and having activity of cleaving the bond between positions 20 and 22 of a sterol side chain, wherein the maximum velocity (Vmax) used as a reaction rate parameter of the aforementioned activity is 40 mmol/min/mol or more; a ferredoxin protein having electron-transferring activity on the enzyme protein; and a ferredoxin reductase protein having electron-transferring activity on the ferredoxin protein are allowed to bind to one another, so that they can function as a single protein,

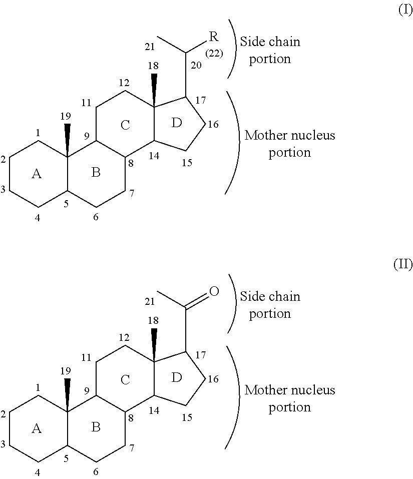

[wherein the definitions of the formula (I) and the formula (II) are the same as those of <1>].
<10> The method according to <8> or <9>, which comprises allowing 3-hydroxysteroid dehydrogenase/isomerase, steroid 17α-hydroxylase, steroid 21-hydroxylase and steroid 11β-hydroxylase to further come into contact with the compound of the formula (II), so as to generate hydrocortisone.
<11> The method according to any one of <8> to <10>, wherein the sterol represented by the above formula (I) is cholesterol, 4-cholesten-3-one, 7-dehydrocholesterol, ergosterol, β-sitosterol, stigmasterol, campesterol, desmosterol, (20S)-20-hydroxycholest-4-en-3-one, (22R)-22-hydroxycholest-4-en-3-one, (20R,22R)-20,22-dihydroxycholest-4-en-3-one, or (20R,22S)-20,22-dihydroxycholest-4-en-3-one.
<12> The method according to any one of <8> to <11>, wherein the compound represented by the above formula (II) is pregnenolone, progesterone, or 7-dehydropregnenolone.
<13> The method according to any one of <8> to <12>, wherein the sterol represented by the above formula (I) is generated by allowing a raw material selected from among glucose, glycerol, methanol, ethanol, sucrose, acetic acid and citric acid, to come into contact with a yeast having ability to assimilate the raw material so as to generate the sterol represented by the above formula (I).
<14> A method for producing hydrocortisone from a raw material selected from among glucose, glycerol, methanol, ethanol, sucrose, acetic acid and citric acid, wherein the method comprises culturing, in a medium containing the aforementioned raw material, a yeast having activity of generating a sterol represented by formula (I) as shown below from the aforementioned raw material, having activity of generating sterol 3beta-hydroxysteroid dehydrogenase and/or 3beta-hydroxysteroid:oxygen oxidoreductase, steroid 17α-hydroxylase, steroid 21-hydroxylase and steroid 11β-hydroxylase, and also having any one of the following characteristics (A) to (E):
(A) having activity of generating a mixture of the enzyme protein according to <1> or <2>, a ferredoxin protein having electron-transferring activity on the enzyme protein, and a ferredoxin reductase protein having electron-transferring activity on the ferredoxin protein;
(B) having activity of generating a mixture of a protein consisting of the amino acid sequence shown in SEQ ID NO: 23, a ferredoxin protein having electron-transferring activity on the aforementioned protein, and a ferredoxin reductase protein that transfers electron to the ferredoxin protein;
(C) having activity of generating a mixture of a protein consisting of an amino acid sequence comprising deletion, substitution and/or addition of one or several amino acids with respect to the amino acid sequence shown in SEQ ID NO: 23, and having activity of cleaving the bond between positions 20 and 22 of a sterol side chain, wherein the maximum velocity (Vmax) used as a reaction rate parameter of the aforementioned activity is 40 mmol/min/mol or more; a ferredoxin protein having electron-transferring activity on the aforementioned protein; and a ferredoxin reductase protein that transfers electron to the ferredoxin protein;
(D) having activity of generating a fusion protein, wherein a protein consisting of the amino acid sequence shown in SEQ ID NO: 23 or a protein consisting of an amino acid sequence comprising a deletion, substitution and/or addition of one or several amino acids with respect to the amino acid sequence shown in SEQ ID NO: 23, and having activity of cleaving the bond between positions 20 and 22 of a sterol side chain, wherein the maximum velocity (Vmax) used as a reaction rate parameter of the aforementioned activity is 40 mmol/min/mol or more; a ferredoxin protein having electron-transferring activity on the enzyme protein; and a ferredoxin reductase protein having electron-transferring activity on the ferredoxin protein are allowed to bind to one another, so that they can function as a single protein; and
(E) having activity of generating a fusion protein, wherein the enzyme protein according to <1> or <2>, a ferredoxin protein having electron-transferring activity on the enzyme protein, and a ferredoxin reductase protein having electron-transferring activity on the ferredoxin protein are allowed to bind to one another, so that they can function as a single protein,

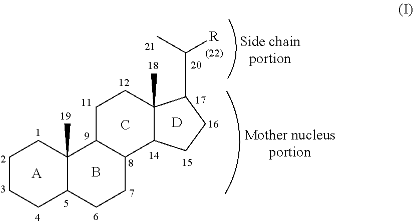

[wherein the definitions of the formula (I) are the same as those of <1>].

### Advantages of the Invention

Using a novel enzyme protein provided by the present invention, which has activity of cleaving the bond between positions 20 and 22 of a sterol compound side chain, it became possible to efficiently produce pregnenolone and the like and hydrocortisone, which are compounds industrially useful as medicaments and pharmaceutical intermediates. In general, when animal-derived P450scc is allowed to express in a microorganism as a host, it is obtained in the form of an insoluble protein, and thus, it is difficult to obtain the protein as an active body. However, when microorganism-derived P450scc provided by the present invention for the first time is allowed to express in a microorganism as a host, it can be obtained in the form of a soluble protein. Thus, since the protein can easily be obtained as an active body, it has high industrial usefulness.

### BEST MODE FOR CARRYING OUT THE INVENTION

Hereinafter, the embodiments of the present invention will be described in detail.

### (1) Protein of the Present Invention

According to the present invention, there is provided a protein described in any one of the following (a), (b) and (c) (hereinafter referred to as “CYPSS204A” at times):

(a) a protein consisting of the amino acid sequence shown in SEQ ID NO: 2;
(b) a protein consisting of an amino acid sequence comprising a deletion, substitution and/or addition of one or several amino acids with respect to the amino acid sequence shown in SEQ ID NO: 2, and having activity of cleaving the bond between positions 20 and 22 of a sterol side chain, wherein the maximum velocity (Vmax) used as a reaction rate parameter of the aforementioned activity is 50 mmol/min/mol or more; and
(c) a protein consisting of an amino acid sequence having homology of 95% or more with the amino acid sequence shown in SEQ ID NO: 2, and having activity of cleaving the bond between positions 20 and 22 of a sterol side chain, wherein the maximum velocity (Vmax) used as a reaction rate parameter of the aforementioned activity is 50 mmol/min/mol or more.

Preferably, with regard to the activity of cleaving the bond between positions 20 and 22 of a sterol side chain of the protein of the present invention, the activity decrease rate is 30% or less, when the substrate concentration is increased from 10 μM to 100 μM.

In the present invention, the term “one or several” in the expression “an amino acid sequence comprising a deletion, substitution and/or addition of one or several amino acids” means, for example, approximately 1 to 20, preferably approximately 1 to 10, and more preferably approximately 1 to 5.

The enzyme protein of the present invention also has the following physicochemical properties.

Action: The enzyme acts on sterol represented by formula (I) as shown below and cleaves the carbon-carbon bond between positions 20 and 22 of the sterol side chain portion by its activity of cleaving the bonds, so as to generate a compound represented by formula (II) as shown below.
Substrate specificity: When microorganisms that produce the enzyme are allowed to react with an aqueous solution containing 100 μg/ml 4-cholesten-3-one or cholesterol at 28° C. for 5 hours, the conversion reaction rate from 4-cholesten-3-one to progesterone is 10% or more (preferably 30% or more, more preferably 50% or more, further preferably 60% or more, and particularly preferably 70% or more), and the conversion reaction rate from cholesterol to pregnenolone is 10% or more (preferably 30% or more, more preferably 50% or more, further preferably 60% or more, and particularly preferably 65% or more).

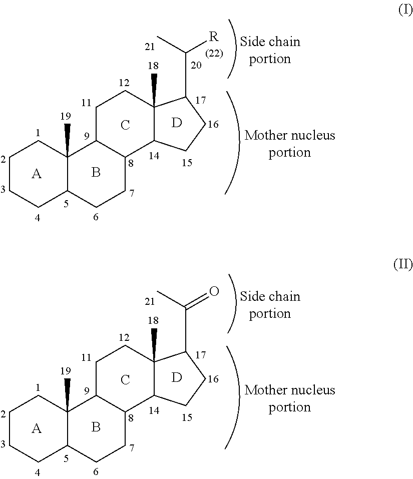

[wherein, in the formula (I), the mother nucleus portion has a structure in which it consists of the A, B, C and D rings of a steroid, wherein the mother nucleus portion has a carbon-carbon unsaturated bond(s) at zero site or at one or more sites of the positions 1 to 17 of the rings (except for the positions 10 and 13 thereof), and carbon(s) at zero site or at one or more sites of the positions 1 to 19 of the rings (except for the positions 10 and 13 thereof) are independently substituted with a group represented by the formula —OX (namely, a hydroxy group wherein X═H; an acyloxyl group wherein X═COR_{I} [wherein R₁ is a hydrogen atom, or an alkyl group, alkenyl group, alkynyl group or aromatic hydrocarbon containing 10 or less carbon atoms]; an O-alkyl group wherein X═R₂ [wherein R₂ is an alkyl group, alkenyl group or alkynyl group containing 10 or less carbon atoms, which may be optionally substituted with an oxygen atom]; a sulfate wherein X═SO₃M [wherein M is a hydrogen atom, an alkaline metal or an alkaline-earth metal]; an O-glycosyl group wherein X is the carbon at position 1 of a sugar; or an epoxy group wherein X is a carbon atom adjacent to said carbon), or a keto group represented by the formula ═O, and

in the side chain portion, R is a linear alkyl group, alkenyl group or alkynyl group containing 10 or less carbon atoms, which may have a cyclic portion, or a branched alkyl group, alkenyl group or alkynyl group containing 10 or less carbon atoms, which may have a cyclic portion, the carbons at positions 20 and 21, and at zero site or at one or more sites in the R, are independently substituted with a group represented by the formula —OY (namely, a hydroxy group wherein Y═H; an acyloxyl group wherein Y═COR₃ [wherein R₃ is a hydrogen atom, or an alkyl group, alkenyl group, alkynyl group or aromatic hydrocarbon containing 10 or less carbon atoms]; an O-alkyl group wherein Y═R₄ [wherein R₄ is an alkyl group, alkenyl group or alkynyl group containing 10 or less carbon atoms, which may be optionally substituted with an oxygen atom]; a sulfate wherein Y═SO₃M [wherein M is a hydrogen atom, an alkaline metal or an alkaline-earth metal]; an O-glycosyl group wherein Y is the carbon at position 1 of a sugar; or an epoxy group wherein Y is a carbon atom adjacent to said carbon), or a keto group represented by the formula ═O], and

[wherein, in the formula (II), the mother nucleus portion has the same definitions as those of the mother nucleus portion of the formula (I), and

in the side chain portion, the carbon at position 21 is substituted with zero or one group represented by the formula —OY (namely, a hydroxy group wherein Y═H; an acyloxyl group wherein Y═COR₃ [wherein R₃ is a hydrogen atom, or an alkyl group, alkenyl group, alkynyl group or aromatic hydrocarbon containing 10 or less carbon atoms]; an O-alkyl group wherein Y═R₄ [wherein R₄ is an alkyl group, alkenyl group or alkynyl group containing 10 or less carbon atoms, which may be optionally substituted with an oxygen atom]; a sulfate wherein Y═SO₃M [wherein M is a hydrogen atom, an alkaline metal or an alkaline-earth metal]; or an O-glycosyl group wherein Y is the carbon at position 1 of a sugar), or zero or one keto group represented by the formula ═O].

The sterol of the formula (I) is preferably cholesterol, 4-cholesten-3-one, 7-dehydrocholesterol, ergosterol, β-sitosterol, stigmasterol, campesterol, desmosterol, (20S)-20-hydroxycholest-4-ene-3-one, (22R)-22-hydroxycholest-4-ene-3-one, (20R,22R)-20,22-dihydroxycholest-4-ene-3-one, or (20R,22S)-20,22-dihydroxycholest-4-ene-3-one; and more preferably cholesterol, 4-cholesten-3-one, or 7-dehydrocholesterol.

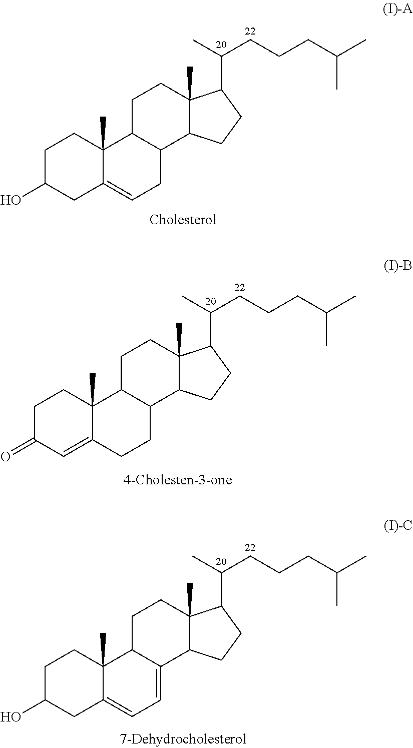

The compound of the formula (II) is preferably pregnenolone, progesterone, or 7-dehydropregnenolone.

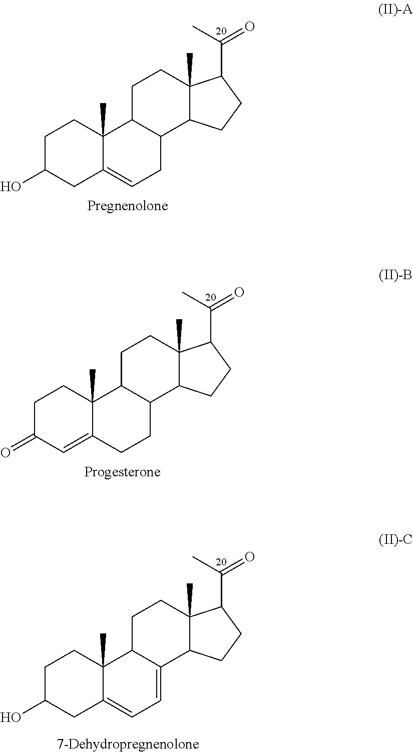

As other properties of the protein of the present invention, the conversion rate from 7-dehydrocholesterol to 7-dehydropregnenolone is 10% or more, preferably 20% or more, and more preferably 25% or more; the conversion rate from ergosterol to 7-dehydropregnenolone is 1% or more, and preferably 2% or more; the conversion rate from β-sitosterol to pregnenolone is 10% or more, preferably 20% or more, more preferably 30% or more, and further preferably 40% or more; and the conversion rate from stigmasterol to pregnenolone is 1% or more, preferably 2% or more, and more preferably 5% or more. Moreover, the conversion rate from campesterol to pregnenolone is 10% or more, preferably 20% or more, and more preferably 25% or more; the conversion rate from desmosterol to pregnenolone is 10% or more, preferably 30% or more, more preferably 50% or more, further preferably 60% or more, and particularly preferably 65% or more; the conversion rate from (20S)-20-hydroxycholest-4-en-3-one to progesterone is 10% or more, preferably 20% or more, more preferably 30% or more, and further preferably 40% or more; the conversion rate from (22R)-22-hydroxycholest-4-en-3-one to progesterone is 10% or more, preferably 20% or more, more preferably 30% or more, and further preferably 40% or more; the conversion rate from (20R,22R)-20,22-dihydroxycholest-4-en-3-one to progesterone is 10% or more, preferably 20% or more, more preferably 30% or more, and further preferably 35% or more; and the conversion rate from (20R,22S)-20,22-dihydroxycholest-4-en-3-one to progesterone is 10% or more, preferably 20% or more, more preferably 30% or more, and further preferably 40% or more. Furthermore, the conversion rate of lanosterol is 0%.

The enzyme protein of the present invention preferably may further have the following physicochemical properties. Optimum pH: 7.5 to 8.0. Optimum temperature for action: 15° C. to 20° C. Thermostability: After preservation at 20° C. for 140 hours, 30% or more of enzyme protein activity is maintained. As for molecular weight, the putative molecular weight is assumed to be 53 to 54 KDa based on the amino acid sequence, and it is measured to be 50 to 56 KDa by SDS electrophoresis. In addition, the maximum velocity (Vmax) used as a reaction rate parameter of the aforementioned activity is 50 mmol/min/mol or more, and the activity decrease rate is 30% or less when the substrate concentration is increased from 10 μM to 100 μM.

Vmax and Km can be obtained by conducting the conversion of 4-cholesten-3-one at a substrate concentration of 0.1 to 500 μM and then examining the relationship between the substrate concentration and the activity of the enzyme protein. Moreover, the activity decrease rate when the substrate concentration is increased from 10 μM to 100 μM can also be obtained by conducting the conversion of 4-cholesten-3-one at a substrate concentration of 0.1 to 500 μM and then examining the relationship between the substrate concentration and the activity of the enzyme protein. Specifically, there is prepared 1 ml of a reaction solution, which comprises 220 μmol CYP204A1 or 250 μmol CYPSS204A, 96 μg/ml spinach-derived ferredoxin, 0.1 U/ml spinach-derived ferredoxin reductase protein, 3 U/ml glucose dehydrogenase, 60 mM glucose, 2 mM NADH and 2 mM NADPH. Tris-HCl is then added to the reaction mixture to adjust to pH 7.5. 20 μl of 4-cholesten-3-one used as a substrate, dissolved in DMSO to have a final concentration of 0.1, 0.5, 1, 2, 5, 10, 20, 50, 100 or 500 μM, is added to the reaction mixture. The reaction is initiated by addition of NADH and NADPH, and it can be carried out at 200 rpm at 15° C. for 60 minutes.

The origin of the enzyme protein of the present invention is not particularly limited. It is preferably an enzyme protein derived from microorganisms. Thus, the origin of the present enzyme protein is, for example, Sphingomonas subterranea, and most preferably, Sphingomonas subterranea NBRC16086. The present enzyme protein may also be derived from Novosphingobium aromaticivorans such as Novosphingobium aromaticivorans ATCC 700278, as long as it has the above-described amino acid sequence or the above-described properties.

### (2) Method for Obtaining Enzyme Protein of the Present Invention

The enzyme protein of the present invention can be obtained from microorganisms such as the above-described Sphingomonas subterranea or Novosphingobium aromaticivorans according to ordinary protein extraction and purification methods. Specific examples of the extraction method include: extraction by cell disintegration, such as chopping with scissors, homogenization, a sonic treatment, osmotic shock procedure, and a freezing and thawing method; extraction using a surfactant; and a combined use thereof. Specific examples of the purification method include salting-out using ammonium sulfate (ammonium sulphate), sodium sulfate or the like, centrifugation, dialysis, ultrafiltration, adsorption chromatography, ion exchange chromatography, hydrophobic chromatography, reverse phase chromatography, gel filtration, gel permeation chromatography, affinity chromatography, electrophoresis, zymography, and a combined use thereof.

Alternatively, the below-mentioned DNA that encodes the protein of the present invention is cloned according to a known method, and the obtained clone is then introduced into a suitable host to allow it to express therein, so that the enzyme protein of the present invention can be obtained. For example, based on the information of the nucleotide sequence of a gene encoding the enzyme protein of the present invention as described in the specification, a probe or a primer specific to the gene of the enzyme protein of the present invention is designed. Using the probe or primer, DNA encoding the enzyme protein of the present invention is isolated or amplified from the DNA library (e.g. a genomic DNA library, a cDNA library, etc.) of microorganisms such as Sphingomonas subterranea or Novosphingobium aromaticivorans. The obtained DNA is introduced into a vector according to a gene recombinant technique, and the vector is then introduced into a host cell, so that it can be expressed therein, thereby obtaining the enzyme protein of the present invention.

### (3) DNA Encoding Protein of the Present Invention, Recombinant Vector, and Transformant

According to the present invention, there is provided DNA encoding the above-described protein of the present invention. A specific example of the DNA of the present invention is DNA described in any one of the following (a), (b) and (c):

(a) DNA having the nucleotide sequence shown in SEQ ID NO: 1;
(b) DNA having a nucleotide sequence comprising a deletion, substitution and/or addition of one or several nucleotides with respect to the nucleotide sequence shown in SEQ ID NO: 1, and encoding a protein which has activity of cleaving the bond between positions 20 and 22 of a sterol side chain, wherein the maximum velocity (Vmax) used as a reaction rate parameter of the aforementioned activity is 50 mmol/min/mol or more; and
(c) DNA having a nucleotide sequence capable of hybridizing under stringent conditions with DNA having the nucleotide sequence shown in SEQ ID NO: 1 or a complementary sequence thereof, and encoding a protein which has activity of cleaving the bond between positions 20 and 22 of a sterol side chain, wherein the maximum velocity (Vmax) used as a reaction rate parameter of the aforementioned activity is 50 mmol/min/mol or more.

A gene encoding the enzyme protein of the present invention can also be isolated from the a DNA library of microorganisms such as Sphingomonas subterranea and Novosphingobium aromaticivorans by performing hybridization using, as a probe, an oligonucleotide produced based on the nucleotide sequence of Sphingomonas subterranea, Novosphingobium aromaticivorans or the like, or by performing PCR using the oligonucleotide produced based on the aforementioned sequence as a primer. Moreover, the gene of the enzyme protein of the present invention may be not only a gene isolated from naturally-existing microorganisms, but also a gene that is synthesized by changing a codon such that the present enzyme protein can be appropriately expressed without the change of the amino acid sequence in a host cell for expressing the enzyme protein.

In the present invention, the term “one or several” in the expression “a nucleotide sequence comprising a deletion, substitution and/or addition of one or several nucleotides” means, for example, approximately 1 to 30, preferably approximately 1 to 20, more preferably approximately 1 to 10, and further preferably approximately 1 to 5. In the present invention, the expression “a nucleotide sequence capable of hybridizing under stringent conditions with . . . ” means DNA and the like comprising a nucleotide sequence having homology on BLAST analysis of 90% or more, and more preferably 95% or more to DNA having the nucleotide sequence shown in SEQ ID NO: 1 or a complementary sequence thereof. Moreover, the “hybridization that is carried out under stringent conditions” can be carried out by a method, which comprises carrying out a reaction in an ordinary hybridization buffer at a temperature of 40° C. to 70° C., and preferably 60° C. to 65° C., and then washing the reaction product in a washing solution having a salt concentration of 15 mM to 300 mM, and preferably 15 mM to 60 mM.

A gene having a nucleotide sequence comprising a deletion, substitution and/or addition of one or several nucleotides with respect to the nucleotide sequence shown in SEQ ID NO: 1 can be produced by ordinary mutation operations such as a method using a mutation inducer or site-directed mutagenesis. These mutation operations can be easily carried out using commercially available kits such as Site-Directed Mutagenesis Kit (Takara Shuzo Co., Ltd.) or QuickChange Site-Directed Mutagenesis Kit (manufactured by STRATAGENE).

According to the present invention, there is further provided a recombinant vector having the DNA of the present invention. The recombinant vector means a recombinant vector comprising the DNA of the present invention and an expression control region capable of expressing a protein encoded by the DNA in a host cell. Specifically, it is a vector generally comprising the DNA of the present invention and a promoter suitable for a host microorganism, wherein the 5′-terminal side of the coding region of the DNA of the present invention is ligated downstream of the promoter. The type of such a vector is not particularly limited, as long as it is capable of replicating in a host microorganism. Examples of the vector include a plasmid vector, a shuttle vector, and a phage vector. Specific examples of the plasmid vector include pBR322, pUC18, pHSG298, pUC118, pSTV28, pTWV228, pHY300PLK (manufactured by Takara Shuzo Co., Ltd., etc.), pKK223-3, and pPL-lambda inducible expression vector (manufactured by Pharmacia, etc.). Specific examples of shuttle vectors of Escherichia coli-coryneform group of bacteria include pCRY30 (JP Patent Publication (Kokai) No. 3-210184 A (1991)), pCRY21 (JP Patent Publication (Kokai) No. 2276575 A), pCRY2KE, pCRY2KX, pCRY31, pCRY3KE and pCRY3KX, pCRY2 and pCRY3 (JP Patent Publication (Kokai) No. 1-191686 A (1989)), pAM330 (JP Patent Publication (Kokai) No. 58-67679 A (1983)), pHM1519 (JP Patent Publication (Kokai) No. 58-77895 A (1983)), pAJ655, pAJ611 and pAJ1844 (JP Patent Publication (Kokai) No. 58-192900 A (1983)), pCG1 (JP Patent Publication (Kokai) No. 57-134500 A (1982)), pCG2 (JP Patent Publication (Kokai) No. 58-35197 A (1983)), pCG4 and pCG11 (JP Patent Publication (Kokai) No. 57-183799 A (1982)), and also their derivatives. In addition, an example of the phage vector is a λFixII vector (manufactured by STRATAGENE, etc.).

On the other hand, when Eumycetes such as yeast are used, there can be applied a method of using a vector by which multiple copies are introduced into the chromosome, among chromosome-introduced-type plasmids (Sakai, A. et al., (1991) Biotechnology (NY) 9, 1382-1385), and the use of multicopy plasmids such as a 2-μm plasmid-derived pESC vector (manufactured by STRATAGENE) or a pAUR vector (manufactured by Takara Bio INC.), among non-chromosome-introduced-type plasmids.

As a promoter for the expression of the DNA encoding the enzyme protein of the present invention in yeast used as a host, there can be used a constant expression promoter for the expression of PGK1 gene encoding phoshoglycerate kinase, ADH1 gene encoding alcohol dehydrogenase, GAP1 gene encoding general amino acid permease, etc. Moreover, an inducible promoter such as GAL1/GAL10 (Johnston, M. et al., (1984) Mol. Cell. Biol. 4(8), 1440-1448) can also be used. When these promoters are used, expression is induced by adding galactose in the absence of glucose.

As a promoter for the expression of the DNA encoding the enzyme protein of the present invention, a promoter possessed by a host microorganism can be generally used. However, the type of a promoter is not limited thereto, and all types of promoters may be used, as long as they have nucleotide sequences for initiating the transcription of the gene of the enzyme protein of the present invention. Specific examples of such a promoter include a lactose operon promoter, a tryptophan operon promoter, a λ-phage-derived PL promoter, and a tryptophan-lactose hybrid (tac) promoter (H. A. Bose et al., Proc. Natl. Acad. Sci. U.S.A., Vol. 80, p. 21 (1983)). Among these promoters, an inducible promoter can be used for the purpose of improving expression efficiency. For example, in the case of the above-described lactose operon promoter, gene expression can be induced by adding lactose or isopropyl-β-D-thiogalactoside (IPTG).

According to the present invention, there is further provided a transformant formed by introducing the DNA of the present invention or a recombinant vector into a host cell. The type of such a host cell, into which the DNA of the present invention or a recombinant vector is to be introduced, is not particularly limited. Suitable examples of such a host include bacteria of genus Escherichia, such as Escherichia coli, bacteria of genus Actinomycetes, bacteria of genus Bacillus, bacteria of genus Serratia, bacteria of genus Pseudomonas, bacteria of genus Corynebacterium, bacteria of genus Brevibacterium, bacteria of genus Rhodococcus, bacteria of genus Lactobacillus, bacteria of genus Streptomyces, bacteria of genus Thermus, bacteria of genus Streptococcus, yeast of genus Saccharomyces, yeast of genus Pichia, yeast of genus Kluyveromyces, yeast of genus Candida, yeast of genus Schizosaccharomyces, yeast of genus Debaryomyces, yeast of genus Yarrowia, yeast of genus Cryptococcus, yeast of genus Xanthophyllomyces, mold of genus Aspergillus, mold of genus Mortierella, mold of genus Fusarium, genus Schizochytrium, and genus Thraustochytrium. Preferred host cells include Escherichia coli, Actinomycetes, bacteria of genus Pseudomonas, and yeast of genus Saccharomyces.

Specifically, there can be used Escherichia coli, Bacillus subtilis, Bacillus brevis, Bacillus stearothermophilus, Serratia marcescens, Pseudomonas putida, Pseudomonas aeruginosa, Corynebacterium glutamicum, Brevibacterium flavum, Brevibacterium lactofermentum, Rhodococcus erythropolis, Thermus thermophiles, Streptococcus lactis, Lactobacillus casei, Streptomyces lividans, Saccharomyces cerevisiae, Saccharomyces bayanus, Pichia pastoris, Kluyveromyces lactis, Candida utilis, Candida glabrata, Schizosaccharomyces pombe, Debaryomyces hansenii, Yarrowia lypolitica, Cryptococcus curvatus, Xanthophyllomyces dendrorhous, Aspergillus nigar, Aspergillus oryzae, Mortierella ramanniana, Mortierella bainieri, Mortierella alpina, Cunninghamella elegans, Fusarium fujikuroi, Schizochytrium limacium, Thraustochytrium aureum, etc.

As a method of introducing a gene into the above-described host microorganisms, transformation methods such as the competent cell method [Journal of Molecular Biology, Vol. 53, p. 159 (1970)], the lithium acetate method [Ito, H. et al. J. Bacteriol., Vol. 153, p. 163 (1983)], a spheroplast method [Hinnen, A., et al. Proc. Natl. Acad. Sci. USA, Vol. 75, p. 1929 (1978)] or the pulse wave electrification method [J. Indust. Microbiol., Vol. 5, p. 159 (1990)], the transduction method using phage [E. Ohtsubo, Genetics, Vol. 64, p. 189 (1970)], the conjugal transfer method [J. G. C. Ottow, Ann. Rev. Microbiol., Vol. 29, p. 80 (1975)], the cell fusion method [M. H. Gabor, J. Bacteriol., Vol. 137, p. 1346 (1979)], etc. can be applied. From these methods, a method suitable for host microorganisms may be selected, as appropriate.

In addition to the above-described gene expression method using an expression vector, the gene may also be expressed by a homologous recombination technique whereby the DNA encoding the enzyme protein of the present invention ligated to a promoter is directly introduced into the chromosome of a host microorganism, or by a technique of introducing the gene using transposon, or the insertion sequence or the like. Accordingly, the transformant of the present invention is sufficient as long as the enzyme protein of the present invention is expressed therein. The method of introducing the gene into a host is not limited.

### (4) Production of Enzyme Protein of the Present Invention Using Transformant of the Present Invention

According to the present invention, a transformant obtained as described in (3) above may be cultured, and the enzyme protein of the present invention may be then collected from the culture.

The transformant can be cultured in an ordinary nutritive medium containing a carbon source, a nitrogen source, inorganic salts, various types of vitamins, etc. Examples of the carbon source used herein include: sugars such as glucose, sucrose, fructose or maltose; alcohols such as ethanol or methanol; organic acids such as citric acid, malic acid, succinic acid, maleic acid or fumaric acid; and blackstrap molasses. As a nitrogen source, ammonia, ammonium sulfate, ammonium chloride, ammonium nitrate, urea and the like are used singly or in combination. Examples of the inorganic salts used herein include potassium monohydrogen phosphate, potassium dihydrogen phosphate, and magnesium sulfate. Other than these substances, nutritive substances such as peptone, meat extract, yeast extract, corn steep liquor, casamino acid, and various types of vitamins such as biotin, may be added to the medium.

The culture is generally carried out under aerobic conditions involving aeration stirring or shaking. The culture temperature is not particularly limited, as long as it allows host microorganisms to grow. In addition, the pH applied during the culture is not particularly limited, either, as long as it allows host microorganisms to grow. During the culture, the pH value can be adjusted by addition of acid or alkali.

An enzyme protein can be collected from the culture according to a known collection method, using the activity of the enzyme protein as an indicator. It is not always necessary to purify the enzyme protein to a homogeneous state. The enzyme protein may be purified up to a purification degree that depends on intended use.

As a roughly purified fraction or a purified enzyme protein used in the present invention, a cell mass separated from culture media obtained as a result of the culture of a transformant; a crushed product obtained by crushing culture media or cell mass by means such as ultrasonic wave or friction under pressure; an extract containing the enzyme protein of the present invention, which is obtained by extracting the crushed product with water or the like; a crude preparation of the enzyme protein of the present invention, which is obtained by further performing a treatment such as ammonium sulfate fractionation or column chromatography on the above-described extract; or a purified enzyme protein preparation, may also be used. Furthermore, a product obtained by immobilizing the above-described cell mass, crushed product, extract, roughly purified fraction or purified enzyme protein on a carrier may also be used.

The above-described cell mass, cell mass crushed product, extract, or purified enzyme protein can be immobilized on a carrier according to a known ordinary method of immobilizing such cell mass or the like on suitable carrier such as acrylamide monomer, alginic acid or carrageenan. For example, when the cell mass is immobilized on the carrier, the cell mass, which has just been recovered from the culture or has been washed with suitable buffer such as approximately 0.02 to 0.2 M phosphate buffer (pH 6 to 10), can be used.

### (5) Method for Producing Pregnenolone, Progesterone and 7-Dehydropregnenolone Using Enzyme Protein of the Present Invention and the Like

According to the present invention, the sterol represented by the above formula (I) is allowed to come into contact with (i) a mixture of the above-described enzyme protein (CYPSS204A) of the present invention, the ferredoxin protein having electron-transferring activity on the enzyme protein, and the ferredoxin reductase protein having electron-transferring activity on the ferredoxin protein, or (ii) a fusion protein of the enzyme protein of the present invention, the ferredoxin protein having electron-transferring activity on the enzyme protein, and the ferredoxin reductase protein having electron-transferring activity on the ferredoxin protein, so as to produce the compound represented by the formula (II).

In the present invention, other than the above-described proteins, the sterol of the formula (I) is allowed to come into contact with (a) mixture of the protein (CYP204A1) consisting of the amino acid sequence shown in SEQ ID NO: 23, the ferredoxin protein having electron-transferring activity on the aforementioned protein, and the ferredoxin reductase protein that transfers electron to the ferredoxin protein, (b) a mixture of: the protein consisting of an amino acid sequence comprising deletion, substitution and/or addition of one or several amino acids with respect to the amino acid sequence shown in SEQ ID NO: 23, and having activity of cleaving the bond between positions 20 and 22 of a sterol side chain, wherein the maximum velocity (Vmax) used as a reaction rate parameter of the aforementioned activity is 40 mmol/min/mol or more; the ferredoxin protein having electron-transferring activity on the aforementioned protein; and the ferredoxin reductase protein that transfers electron to the ferredoxin protein, or (c) a fusion protein, wherein a protein consisting of the amino acid sequence shown in SEQ ID NO: 23 or the protein consisting of an amino acid sequence comprising deletion, substitution and/or addition of one or several amino acids with respect to the amino acid sequence shown in SEQ ID NO: 23, and having activity of cleaving the bond between positions 20 and 22 of the sterol side chain, wherein the maximum velocity (Vmax) used as a reaction rate parameter of the aforementioned activity is 40 mmol/min/mol or more; the ferredoxin protein having electron-transferring activity on the enzyme protein; and the ferredoxin reductase protein having electron-transferring activity on the ferredoxin protein are allowed to bind to one another, so that they can function as a single protein, thereby producing the compound represented by the formula (II).

Herein, the ferredoxin protein having electron-transferring activity and the ferredoxin reductase protein having electron-transferring activity on the ferredoxin protein may be a single protein having the activities of the two above proteins. For example, there can be used fusion protein, which is artificially produced by ligating the P450 reductase domain of the P450-BM3 gene of Bacillus megaterium to the enzyme protein (CYP204A1) (Helvig, C. and Capdevila, J. H., Biochemistry, (2000) 39, 5196-5205). Moreover, the above-described protein may be allowed to come into contact with the sterol represented by the above formula (I) by adding the enzyme protein to the sterol, or by allowing microorganisms that produce the above-described protein or a treated product thereof, or the above described transformant or a treated product thereof, to come into contact with the sterol.

With regard to ferredoxin and ferredoxin reductase, the ferredoxin reductase is selected from, for example, the following: spinach-derived ferredoxin reductase, Pseudomonas putida-derived putida redoxin reductase, animal-derived adrenodoxin reductase, Sphingomonas subterranea-derived ferredoxin reductase, Novosphingobium aromaticivorans-derived ferredoxin reductase, Escherichia coli-derived flavodoxin reductase or ferredoxin reductase, Saccharomyces cerevisiae-derived ferredoxin reductase, and other ferredoxin reductases. The ferredoxin is selected from, for example, the following: spinach-derived ferredoxin, Pseudomonas putida-derived putida redoxin, animal-derived adrenodoxin, Sphingomonas subterranea-derived ferredoxin, Novosphingobium aromaticivorans-derived ferredoxin, Escherichia coli-derived flavotoxin or ferredoxin, Saccharomyces cerevisiae-derived ferredoxin, and other ferredoxin-type proteins.

The reaction method is not particularly limited. The sterol represented by the formula (I) used as a substrate is added to the liquid containing the enzyme protein of the present invention (CYPSS204A) and CYP204A1, or microorganisms that produce the aforementioned enzyme protein, etc., and the mixed solution is then reacted at an appropriate temperature such as approximately 10° C. to 40° C. By this reaction, the compound represented by the formula (II), such as pregnenolone, progesterone or 7-dehydropregnenolone, can be produced. Alternatively, even in a case in which microorganisms that produce the enzyme protein of the present invention intrinsically generate the sterol represented by the formula (I), they are reacted so as to produce the compound represented by the formula (II).

A method of fractionating the compound of interest represented by the formula (II) from the reaction mixture is not particularly limited. A separation or purification method known to a person skilled in the art can be applied. The compound of the formula (II) can be fractionated, for example, by solvent extraction, crystallization, resin adsorption, column chromatography, and the like, but the method is not limited thereto.

### (6) Method for Producing Hydrocortisone and Derivative Thereof

In the method of producing the compound represented by the above formula (II), steroid 17α-hydroxylase protein, steroid 21-hydroxylase protein, steroid 11β-hydroxylase protein, 3beta-hydroxysteroid dehydrogenase and/or 3beta-hydroxysteroid:oxygen oxidoreductase protein and sterol Δ7-reductase protein are allowed to further come into contact with the compound of the formula (II), so that hydrocortisone can be produced.

The steroid 17α-hydroxylase protein, the steroid 21-hydroxylase protein, the steroid 11β-hydroxylase protein, the 3beta-hydroxysteroid dehydrogenase and/or 3beta-hydroxysteroid:oxygen oxidoreductase protein and the sterol Δ7-reductase protein may be allowed to come into contact with the compound represented by the above formula (II) or a product converted from the compound of the formula (II) as a result of the enzyme reaction of any one of the aforementioned proteins, by adding the enzyme proteins to the compound, or by allowing microorganisms that produce the protein or a treated product thereof, or the above described transformant or a treated product thereof, to come into contact with the compound. The steroid 17α-hydroxylase protein, the steroid 21-hydroxylase protein, the steroid 11β-hydroxylase protein and the 3beta-hydroxysteroid dehydrogenase and/or 3beta-hydroxysteroid:oxygen oxidoreductase protein, which are used herein, may be those described in Molecular and Cellular Endocrinology 1990, 73, 73-80, for example. As a sterol Δ7-reductase protein, a protein derived from Arabidopsis thaliana, described in Journal of Biological Chemistry 1996, 271, 10866-10873, or a protein derived from mold of genus Mortierella, described in Applied and Environmental Microbiology 2007, 73, 1736-1741, is used. As a steroid 17α-hydroxylase protein, cytochrome P450c17 derived from cattle, mouse, rat or human being is preferable, for example. Moreover, as a steroid 21-hydroxylase protein, cytochrome P450c21 derived from cattle, mouse, rat or human being is preferable. As a steroid 11β-hydroxylase protein, cytochrome P450c11 derived from cattle, mouse, rat or human being, Curvularia lunata-derived P-450lun, and the like are preferable. Furthermore, as a 3beta-hydroxysteroid dehydrogenase and/or 3beta-hydroxysteroid:oxygen oxidoreductase protein, 3β-hydroxysteroid dehydrogenase (3βHSD) derived from cattle, mouse, rat or human being, and cholesterol oxidase derived from bacteria of genus Streptomyces are preferable. As a sterol A7-reductase protein, Arabidopsis thaliana-derived NADPH-sterol Δ7-reductase and Mortierella alpina-derived sterol Δ7-reductase (MoDELTA7SR) are preferable.

Furthermore, the method for producing hydrocortisone of the present invention preferably includes a method for producing hydrocortisone from a raw material selected from among glucose, sucrose, glycerol, methanol, ethanol, citric acid and acetic acid, wherein the method comprises culturing, in a medium containing the aforementioned raw material, a yeast or mold having activity of generating the sterol represented by the above formula (I) from the aforementioned raw material, also having activity of generating sterol Δ7-reductase, 3beta-hydroxysteroid dehydrogenase and/or 3beta-hydroxysteroid:oxygen oxidoreductase, steroid 17α-hydroxylase, steroid 21-hydroxylase and steroid 11β-hydroxylase, and also having any one of the following characteristics (A) to (E):

(A) having activity of generating a mixture of the enzyme protein according to claim 1 or 2, the ferredoxin protein having electron-transferring activity on the enzyme protein, and the ferredoxin reductase protein having electron-transferring activity on the ferredoxin protein;
(B) having activity of generating a fusion protein, wherein the enzyme protein according to claim 1 or 2, the ferredoxin protein having electron-transferring activity on the enzyme protein, and the ferredoxin reductase protein having electron-transferring activity on the ferredoxin protein are allowed to bind to one another, so that they can function as a single protein;
(C) having activity of generating a mixture of the protein consisting of the amino acid sequence shown in SEQ ID NO: 23, the ferredoxin protein having electron-transferring activity on the aforementioned protein, and the ferredoxin reductase protein that transfers electron to the ferredoxin protein;
(D) having activity of generating a mixture of: the protein consisting of an amino acid sequence comprising deletion, substitution and/or addition of one or several amino acids with respect to the amino acid sequence shown in SEQ ID NO: 23, and having activity of cleaving the bond between positions 20 and 22 of the sterol side chain, wherein the maximum velocity (Vmax) used as a reaction rate parameter of the aforementioned activity is 40 mmol/min/mol or more; the ferredoxin protein having electron-transferring activity on the aforementioned protein; and the ferredoxin reductase protein that transfers electron to the ferredoxin protein; and
(E) having activity of generating fusion protein, wherein the protein consisting of the amino acid sequence shown in SEQ ID NO: 23 or the protein consisting of an amino acid sequence comprising deletion, substitution and/or addition of one or several amino acids with respect to the amino acid sequence shown in SEQ ID NO: 23, and having activity of cleaving the bond between positions 20 and 22 of the sterol side chain, wherein the maximum velocity (Vmax) used as a reaction rate parameter of the aforementioned activity is 40 mmol/min/mol or more; the ferredoxin protein having electron-transferring activity on the enzyme protein; and the ferredoxin reductase protein having electron-transferring activity on the ferredoxin protein are allowed to bind to one another, so that they can function as a single protein.

In the present invention, with regard to the description “having activity of generating the protein,” there is preferably used a method, which involves the insertion of DNA encoding the protein into the above-described expression vector and the use of a transformed yeast or a transformed mold obtained by transforming the above-described yeast or mold.

Examples of the yeast or mold that is preferably used herein include yeast of genus Saccharomyces, yeast of genus Pichia, yeast of genus Schizosaccharomyces, yeast of genus Yarrowia, mold of genus Aspergillus, and mold of genus Mortierella. A more preferred example is yeast of genus Saccharomyces. Specifically, it is Saccharomyces cerevisiae. More specific examples of the Saccharomyces cerevisiae include Saccharomyces cerevisiae (JP2004-528827 A1) strain, FY1679-18b strain (JP2004-528827 A1), KA311A strain (FERM:P-19053), YPH499 strain (ATCC#204679), and YPH500 strain (ATCC#204680). DNA encoding each enzyme protein is preferably inserted into an expression vector such as pESC-LEU (STRATAGENE) or the chromosome of the yeast. In addition, these yeasts or molds preferably have any one or all of the following properties (1) to (6): (1) the site between positions 22 and 23 of intrinsic sterol cannot be unsaturated by modifying the DNA sequence of the gene encoding a sterol-22 desaturase protein or a region that controls the expression thereof; (2) the expression of an NADP-cytochrome P450 reductase protein is reinforced by introducing one or more copies of gene encoding the present protein and an expression control region thereof into host cells; (3) intrinsic sterol cannot be esterified by modifying the DNA sequence of a gene encoding sterol esterification enzyme protein or a region that controls the expression thereof; (4) the expression of sterol ester hydrolase is reinforced by introducing one or more copies of gene encoding the present protein and an expression control region thereof into host cells; (5) by introducing gene encoding each of ferredoxin-type protein that transfers electron to steroid 17α-hydroxylase, steroid 21-hydroxylase and steroid 11β-hydroxylase, and ferredoxin reductase protein having electron-transferring activity on the aforementioned protein, and an expression control region thereof into host cells, the expression of the aforementioned proteins is reinforced; and (6) as a result of modification of the DNA sequence of the gene encoding a sterol-24 methyl transferase protein or a region that controls the expression thereof, no methyl groups are present in the position 24 of intrinsic sterol.

A method of fractionating hydrocortisone from the reaction mixture is not particularly limited, and a separation or purification method known to a person skilled in the art can be applied. Hydrocortisone can be fractionated, for example, by solvent extraction, crystallization, resin adsorption, column chromatography, and the like, but the method is not limited thereto.

The derivatives shown in FIGS. 2 and 3 can be produced from the above-produced hydrocortisone according to a known method. Moreover, the thus produced hydrocortisone and derivatives thereof are screened in terms of activity or safety as medicaments by in vitro or in vivo pharmacological or physiological tests according to known methods, and as a result, they can be used as therapeutic agents for diseases. In this case, a method of producing a pharmaceutical composition comprising, as an active ingredient, the compound obtained in the above-described process is also included in the present invention.

Specifically, the compound may be used alone, but it may also be mixed with a pharmaceutically acceptable carrier and may be then used as a pharmaceutical composition. The thus obtained pharmaceutical compound may be used alone, but it may also be mixed with a pharmaceutically acceptable carrier and may be then used as a pharmaceutical composition. The ratio of the active ingredient to the carrier can be fluctuated from 0.01% to 90% by weight.

The pharmaceutical composition obtained in the present invention may be orally administered in a dosage form such as a tablet coated with sugar as necessary, a capsule, a soft capsule, a powder agent, a granule agent, a fine grain agent, a syrup, an emulsion, a suspension or a liquid agent. Alternatively, the present pharmaceutical composition may be processed into an aseptic solution with a pharmaceutically acceptable liquid or an injection such as a suspension, and it may be administered, for example, via intravenous administration, intramuscular administration, local administration, or subcutaneous administration. It is also possible to administer the present pharmaceutical composition via intramucosal administration in the form of a suppository, a sublingual tablet, a transnasal agent, a transpulmonary agent and the like, or in the form of external preparations such as an ointment, a patch and the like. When a composition is prepared for use in oral or parenteral administration, it can be mixed with an organic or inorganic, solid or liquid carrier or diluent, in a unit capacity and a form commonly applied together therewith. The amount of an active ingredient in such a preparation is adjusted, such that an appropriate capacity can be obtained in the designated range.

Examples of an excipient used in the production of a solid preparation for use in oral and transmucosal administration include lactose, sucrose, starch, talc, cellulose, dextrin, kaoline, and calcium carbonate. A liquid preparation for use in oral administration, namely, a syrup, a suspension, a liquid agent or the like, comprises a commonly used inactive diluent, such as water or vegetable oil. This preparation may also comprise auxiliary agents such as a wetting agent, a suspension adjuvant, a sweetener, a flavor, a coloring agent, a preservative and a stabilizer, as well as the aforementioned inactive diluent. Examples of a solvent or a suspending agent used in the production of an injection, a transmucosal agent, etc., include water, propylene glycol, polyethylene glycol, benzyl alcohol, ethyl oleate, and lecithin. Examples of a base used in a suppository include cacao butter, emulsified cacao butter, laurin butter, and Witepsol. In the production of external agents, alcohol, fatty acid esters, propylene glycol, etc. are used as solubilizers or solubilizing agents; a carboxy vinyl polymer, polysaccharide, etc. are used as thickeners; and a surfactant, etc. are used as emulsifiers.

### EXAMPLES

General experimental methods applied in the following examples were carried out in accordance with the experimental manual of Sambrook et al. (Sambrook, J., and Russel, D. W.: Molecular cloning: a laboratory manual. 3rd edition. Cold Spring Harbor Laboratory Press, Cold Spring Harbor, N.Y., 2001).

### Example 1

### Obtainment of DNA Encoding Sphingomonas subterranea NBRC16086-Derived P450scc (CYPSS204A) and Ferredoxin

Sphingomonas subterranea NBRC 16086 (Biological Resource Center, Biotechnology Field, National Institute of Technology and Evaluation, Incorporated Administrative Agency) was inoculated into an L medium (1% tryptone, 0.5% yeast extract, 0.5% NaCl, 0.1% glucose, pH 7.2), and it was then cultured at 30° C. overnight. Thereafter, genomic DNA was extracted from the obtained cell mass. The genomic DNA was extracted using a DNA extraction kit ISOPLANT (manufactured by Wako Pure Chemical Industries, Ltd.) A portion of DNA encoding CYPSS (hereinafter referred to as a “CYPSS204A gene” at times) was amplified from the genomic DNA by a polymerase chain reaction (PCR) using a primer CYP-1F (SEQ ID NO: 5) and a primer CYP-2R (SEQ ID NO: 6) and using LA Taq polymerase (manufactured by Takara Bio INC.). The temperature conditions applied during the PCR were 94° C./3 min, 30 cycles of (94° C./30 sec, 55° C./30 sec, and 72° C./90sec), and 72° C./5 min.

The nucleotide sequence of a 1.0-kb gene fragment amplified as a result of the above-described reaction was determined. Based on the obtained nucleotide sequence information, inverse PCR was carried out, and the entire nucleotide sequence (SEQ ID NO: 3) of a 1.8-kb DNA region comprising the above-described CYP204A1 gene and a ferredoxin gene located downstream thereof was determined. The nucleotide sequence of the CYPSS204A gene (nucleotides 1 to 1419 of SEQ ID NO: 3) is shown in SEQ ID NO: 1, and the amino acid sequence of CYPYP204A1 is shown in SEQ ID NO: 2. In the nucleotide sequence shown in SEQ ID NO: 1, nucleotides 1430 to 1762 correspond to the ferredoxin.

### Example 2

### Production of Escherichia coli Expression Strain BP215 that Expresses Sphingomonas subterranea NBRC 16086-Derived P450scc (CYPSS204A)

A DNA region comprising CYPSS204A and a ferredoxin gene located downstream thereof was amplified from the Sphingomonas subterranea NBRC 16086 genomic DNA obtained in Example 1 by PCR using a primer CYP-3F (SEQ ID NO: 7) and a primer CYP-4R (SEQ ID NO: 8) and also using KOD plus polymerase (manufactured by TOYOBO Co., Ltd.). The temperature conditions applied during the PCR were 95° C./5 min, 30 cycles of (98° C./30 sec, 60° C./30 sec, and 68° C./2 min), and 68° C./10 min.

The amplified 1.8-kb DNA fragment was purified using QIAquick PCR purification Kit (manufactured by QIAGEN), and the resultant was then digested with Nde I and Spe I. Thereafter, using T4 DNA ligase, the digest was ligated to an Escherichia colit expression vector pT7NS-camAB (International Publication WO2003/087381, pamphlet), and Escherichia coli DH5alpha was then transformed with the expression vector, so as to construct a plasmid pCYPSS-camAB. Escherichia coli BL21(DE3) was transformed with this plasmid, and the obtained cell strain was named as BP215.

### Example 3

### Obtainment of DNA Encoding P450scc (CYP204A1) from Novosphingobium aromaticivorans ATCC 700278 and Production of Escherichia coli Expression Strain BP172

Novosphingobium aromaticivorans (ATCC 700278) was inoculated into an L medium, and it was then cultured at 30° C. for 2 days. Thereafter, genomic DNA was extracted from the cell mass obtained in the same manner as that of Example 1. A fragment comprising a CYPSS204A1 gene and a ferredoxin gene located downstream thereof was amplified from the above-described genomic DNA by using a primer CYP-5F (SEQ ID NO: 9) and a primer CYP-6R (SEQ ID NO: 10) and also using La Taq polymerase (manufactured by Takara Bio INC.). The temperature conditions applied during this operation were 94° C./3 min, 30 cycles of (98° C./20 sec, 63° C./30 sec, and 68° C./2 min), and 72° C./5 min. The nucleotide sequence of the amplified 1.8-kb DNA product is shown in SEQ ID NO: 4. In this nucleotide sequence, nucleotides 1 to 1422 correspond to the CYP204A1, and nucleotides 1433 to 1765 correspond to the ferredoxin gene.

A 1.8-kb DNA fragment amplified as a result of this reaction was purified using QIAquick PCR purification Kit (manufactured by QIAGEN), and the resultant was then digested with Nde I and Spe I. Using T4 DNA ligase, the digest was ligated to an Escherichia coli expression vector pT7-camAB, and Escherichia coli DH5alpha was then transformed with the expression vector, so as to construct a plasmid pCYP204A1-camAB. Escherichia coli BL21(DE3) was transformed with this plasmid, and the obtained cell strain was named as BP172. The amino acid sequence encoded by the CYP204A1 gene is shown n SEQ ID NO: 23.

### Example 4

### Expression of CYP204A1 Gene in Bacillus megaterium and Conversion of 4-cholesten-3-one to Progesterone with Use of this Cell Mass

The pCYP204A1-camAB produced in Example 3 was used as a template, and a fragment comprising the CYP204A1 gene and the ferredoxin gene located downstream thereof was amplified using primers CYP-7F (SEQ ID NO: 11) and CYP-8R (SEQ ID NO: 12) and KOD plus polymerase (manufactured by TOYOBO Co., Ltd.). The temperature conditions applied during this operation were 94° C./2 min, 20 cycles of (94° C./20 sec, 55° C./30 sec, and 68° C./3 min), and 72° C./5 min.

A 1.8-kb DNA fragment amplified as a result of this reaction, which comprised the CYP204A1 gene and the ferredoxin gene located downstream thereof, was purified using QIAquick PCR purification Kit (manufactured by QIAGEN), and the resultant was then digested with Nhe I and Bam HI. Thereafter, using T4 DNA ligase, the digest was ligated to Bacillus megaterium expression vector pHW1520 (manufactured by MoBiTec), and Escherichia coli DHSalpha was then transformed with the expression vector, so as to construct a plasmid pXYL204CB. Bacillus megaterium competent cells (manufactured by MoBiTec) were transformed with this plasmid, so as to obtain a cell strain pXYL204CB.

This cell strain was inoculated into 2 ml of TB medium containing tetracycline (final concentration: 10 mg/ml), and it was then subjected to shaking culture at 37° C. at 220 rpm for 16 hours. Thereafter, 250 μl of the obtained pre-culture was added to 25 ml of the same type of TB medium, and it was then subjected to shaking culture at 37° C. for 2.5 hours. Subsequently, 250 μl of 50% xylose was added to the culture solution, and the mixture was further subjected to a shaking culture at 30° C. at 125 rpm for 6 hours. The cell mass recovered from the obtained culture by centrifugation was suspended in 5 ml of buffer for conversion (50 mM KPB, 2% glycerol [pH 7.4]) to prepare a cell suspension.

Thereafter, 2 μl of solution of 4-cholesten-3-one in methanol (100 mg/ml) was added to 2 ml of the cell suspension, and the obtained mixture was then incubated at 30° C. for 24 hours while shaking (220 rpm). Then, 2 ml of ethyl acetate was added to the reaction solution, and the obtained mixture was then shaken with the use of a vortex, followed by centrifugation at 4,000 rpm for 10 minutes. The obtained ethyl acetate phase (1.5 ml) was solidified by concentration, it was then dissolved in 200 μl of methanol, and the obtained solution was then subjected to HPLC analysis. As a result of the analysis, it was found that 4-cholesten-3-one was converted to progesterone at a conversion rate of 24.5%. HPLC conditions were as follows: Column: Inertsil ODS-3 4.6×50 mm, flow rate: 1.2 ml/min, temp.: 40° C., detect: PDA (240 nm), gradient: 0/4/5/5.5 (min), 40/100/100/40 (% B).

### Example 5

### Properties of Enzyme Protein

### (1) Construction of Expression Vector

In order to amplify DNA fragments each comprising a CYP204A1, CYPSS or CYP11A gene, the following primers were produced: CYP204A1-1F (SEQ ID NO: 13), CYP204A1-2R (SEQ ID NO: 14), CYPSS-1F (SEQ ID NO: 15), CYPSS-2R (SEQ ID NO: 16), CYP11A-1F (SEQ ID NO: 17), CYP11A-2R (SEQ ID NO: 18). Subsequently, pCYP204A1-camAB, pCYPSS-camAB, and pCYP11A-ARX (see Reference Example 1) into which CYP11A had been inserted as bovine-derived P450scc, were used as templates, and PCR reaction was carried out using the aforementioned primers and KOD plus (TOYOBO). The temperature conditions applied during this reaction were 95° C./3 min, 25 cycles of (98° C./20 sec, 55° C./30 sec, and 72° C./2 min), and 72° C./5 min.

As a result of this reaction, approximately 1.4-kbp DNA fragments (hereinafter referred to as DNA fragments A, B and C, respectively) were amplified from CYP204A1, CYPSS and CYP11A. These DNA fragments were purified using Wizard SV Gel and PCR Clean-Up System (PROMEGA) and were then digested with NdeI and XhoI. Then, using DNA Ligation Kit ver 2.1 (TaKaRa), each digest was ligated to pET22b, so as to obtain plasmids pET22-CYP204A1, pET22-CYPSS, and pET22-CYP11A.

### (2) Production of CYP204A1-, CYPSS204A- and CYP11A-Induced Cell Masses

Escherichia colt BL21 (DE3) was transformed with each of the plasmids pET22-CYP204A1, pET22-CYPSS, and pET22-CYP11A prepared in (1) above. Each of the thus obtained transformants was inoculated into 1 ml of TB medium containing 50 μg/ml carbenicillin, and it was then cultured at 37° C. for 14 hours. Thereafter, 1 ml of the culture solution was inoculated into 100 ml of TB medium containing 50 μg/ml carbenicillin and Overnight Express Autoinduction system 1 (Merck), followed by a culture at 25° C. for 24 hours and induction of proteins of interest. Each cell mass was recovered by centrifugation, and it was then suspended in 20 ml of CV buffer (50 mM KPB (potassium phosphate buffer), 10% glycerol, 1 mM EDTA, 2 mM dithiothreitol, and 1 mM D-glucose). Then, the cell suspension was conserved at −80° C.

### (3) Preparation of Cell-Free Extract

To 20 ml of the cell suspension prepared in (2) above, 680 μl of X10 Bug Buster (Novagen), 20 μl of Benzonase (Novagen), and 2 ml of 40 mg/ml lysozyme were added, and the obtained mixture was then shaken at 30° C. for 30 minutes to disintegrate the cells. Thereafter, the reaction solution was centrifuged to obtain the cell-free extract. The obtained, approximately 20 ml of cell-free extract was dialyzed against 1 L of Buffer R (20 mM KPB pH 7.4, 20% glycerol) two times, and the deposited precipitate was then removed by centrifugation. The cell-free extract obtained as a supernatant herein was used in the subsequent experiments.

### (4) Confirmation of Expression of Various Types of P450scc by Spectral Analysis of Carbon Monoxide Binding

The obtained cell-free extract was divided into two portions, and one portion was bubbled with carbon monoxide. Dithionite was added to the two samples, to which carbon monoxide bubbling had been or had not been performed, and difference spectrum was then measured. A peak having maximum absorption around 450 nm was obtained in the case of CYP204A1 and CYPSS204A, and thus, the expression of P450scc was confirmed. On the other hand, in the case of CYP11A, such a peak could not be obtained, and thus, it was suggested that no active P450 would be present in the sample. Based on these results, the subsequent experiments were carried out using CYP204A1 and CYPSS.

### (5) Analysis of the Optimum pH

Using the cell-free extract prepared in (3) above, conversion of 4-cholesten-3-one was carried out at various pH values. The reaction solution was prepared, such that 1 ml of Buffer R comprised 215 μmol/ml CYP204A1 or 367 μmol/ml CYPSS204A, 64 μg/ml spinach-derived ferredoxin, a 0.1 U/ml spinach-derived ferredoxin reductase protein, 3 U/ml glucose dehydrogenase, 60 mM glucose, 50 μg/ml 4-cholesten-3-one, 2 mM NADH, and 2 mM NADPH. Then, 562 μl of 200 mM Buffer having various pH values was added to 1 ml of the reaction solution, so as to adjust the pH of the reaction solution. The reaction was initiated by the addition of NADH and NADPH, and it was carried out at 200 rpm at 30° C. for 14 hours. Thereafter, 1.5 ml of ethyl acetate was added to the reaction solution to terminate the reaction, and extraction was carried out. Then, extraction was carried out again with 0.75 ml of ethyl acetate. The obtained ethyl acetate phase was solidified using evaporator, and the residue was then dissolved in 200.d of methanol. Thereafter, progesterone was detected by HPLC analysis. The enzyme protein activity for conversion of 4-cholesten-3-one to progesterone under various pH conditions is shown in Table 1. The enzyme protein activity was indicated by the amount (g) of progesterone generated per hour per mol of P450. The results demonstrated that CYP204A1 and CYPSS had the highest activity at pH 7.5 of Tris-HCl.

### (6) Analysis of the Optimum Temperature

Using the cell-free extract prepared in (3) above, conversion of 4-cholesten-3-one was carried out at various pH values. The reaction solution was prepared, such that 1 ml of the buffer comprised 215 pmol/ml CYP204A1 or 391 pmol/ml CYPSS, 64 μg/ml spinach-derived ferredoxin, 0.1 U/ml spinach-derived ferredoxin reductase protein, 3 U/ml glucose dehydrogenase, 60 mM glucose, 50 μg/ml 4-cholesten-3-one, 2 mM NADH, and 2 mM NADPH. Tris-HCl was added to the reaction solution so as to adjust it to pH 7.5. The reaction was initiated by addition of NADH and NADPH, and it was carried out at 200 rpm for 4 hours, under temperature conditions of 10° C., 15° C., 20° C., 25° C., or 30° C. Thereafter, 1.5 ml of ethyl acetate was added to the reaction solution to terminate the reaction, and extraction was carried out. Then, extraction was carried out again with 0.75 ml of ethyl acetate. The obtained ethyl acetate phase was solidified using evaporator, and the residue was then dissolved in 200 μl of methanol. Thereafter, progesterone was detected by HPLC analysis. The enzyme protein activity for conversion of 4-cholesten-3-one to progesterone under various temperature conditions is shown in Table 1. The results demonstrated that CYP204A1 had the highest activity from 15° C. to 20° C. and CYPSS had the highest activity at 15° C.

TABLE 1
Optimum pH

Specific activity
(g/hr/mol)

| Buffer | pH | CYP204A1 | CYPSS |
|---|---|---|---|
| KPB | 6.2 | 0 | 0 |
| | 6.7 | 0 | 19 |
| | 7.1 | 0 | 21 |
| | 7.4 | 0 | 0 |
| | 7.9 | 0 | 0 |
| Tris-HCl | 7.5 | 207 | 301 |
| | 8.2 | 114 | 269 |
| | 8.7 | 0 | 40 |
| TES | 6.6 | 0 | 0 |
| | 6.8 | 0 | 16 |
| | 7.3 | 39 | 76 |
| | 7.5 | 97 | 165 |
| | 8.0 | 82 | 168 |

Optimum temperature

Specific activity
(g/hr/mol)

| Temp. (° C.) | CYP204A1 | CYPSS |
|---|---|---|
| 10 | 507 | 918 |
| 15 | 698 | 1279 |
| 20 | 719 | 1133 |
| 25 | 550 | 936 |
| 30 | 285 | 465 |

### (7) pH Stability Test

0.5 ml of the cell-free extract prepared in (3) above was mixed with 0.5 ml of 50 mM buffer having various pH values, pH was then measured, and the mixture was then conserved at 6° C. for 140 hours. The pH values of the enzyme protein solution were: AcB (acetate buffer), pH 6.0; KPB, pH 6.7; KPB, pH 7.2; KPB, pH 7.6; TES, pH 7.5; Tris-HCl, pH 8.1; and Tris-HCl, pH 8.9. Using the prepared cell-free extracts having various pH values, conversion of 4-cholesten-3-one was carried out. The reaction was carried out, immediately after the enzyme protein solution was mixed with buffers having various pH values and after the mixed solution was conserved for 140 hours, so as to examine the stability. The reaction solution was prepared, such that 1 ml of the buffer comprised an enzyme protein solution of CYP204A1 or CYPSS having various pH values, 64 μg/ml spinach-derived ferredoxin, 0.1 U/ml spinach-derived ferredoxin reductase protein, 3 U/ml glucose dehydrogenase, 60 mM glucose, 50 μg/ml 4-cholesten-3-one, 2 mM NADH, and 2 mM NADPH. To the reaction solution, 562 μl of 200 mM Tris-HCl was added, so as to adjust the reaction solution to pH 7.5. It was confirmed that the reaction solution maintained pH 7.5, even though enzyme protein solutions having various pH values were added thereto. The reaction was initiated by addition of NADH and NADPH, and it was carried out at 200 rpm at 15° C. for 4 hours. Thereafter, 1.5 ml of ethyl acetate was added to the reaction solution to terminate the reaction, and extraction was carried out. Then, extraction was carried out again with 0.75 ml of ethyl acetate. The obtained ethyl acetate phase was solidified using an evaporator, and the residue was then dissolved in 200 μl of methanol. Thereafter, progesterone was detected by HPLC analysis. The enzyme activity for conversion of 4-cholesten-3-one to progesterone at 0 hour and 140 hours is shown in Table 2. The results demonstrated that both CYP204A1 and CYPSS were most stable at KPB, pH 7.6.

TABLE 2

Activity (μg/ml)

| | | | |
|---|---|---|---|
| Sample | 0 hr | 140 hr | Relative activity (%) |

CYP204A1

| | | | |
|---|---|---|---|
| AcB pH 6.0 | 2.1 | 0.5 | 24 |
| KPB pH 6.7 | 1.9 | 1 | 53 |
| KPB pH 7.2 | 2 | 1.3 | 65 |
| KPB pH 7.6 | 2 | 1.4 | 70 |
| TES pH 7.5 | 2.2 | 1.5 | 68 |
| Tris pH 7.4 | 2.1 | 1.3 | 62 |
| Tris pH 8.1 | 2 | 1 | 50 |
| Tris pH 8.9 | 2 | 0.7 | 35 |

CYPSS

| | | | |
|---|---|---|---|
| AcB pH 6.0 | 7.3 | 2.7 | 37 |
| KPB pH 6.7 | 7.1 | 4.3 | 61 |
| KPB pH 7.2 | 7.3 | 5.3 | 73 |
| KPB pH 7.6 | 6.5 | 5.5 | 85 |
| TES pH 7.5 | 7.3 | 5.2 | 71 |
| Tris pH 7.4 | 8 | 5 | 63 |
| Tris pH 8.1 | 6.9 | 4.5 | 65 |
| Tris pH 8.9 | 7.3 | 3.7 | 51 |

### (8) Thermostability Test

The cell-free extract prepared in (3) above was conserved at various temperature for 140 hours. The conservation temperature was set at 6° C., 10° C., 20° C., 30° C., 37° C., or 45° C. Using the enzyme protein solution before conservation and the sample obtained after conservation for 140 hours, conversion of 4-cholesten-3-one was carried out, and stability was examined. The reaction solution was prepared, such that 1 ml of the buffer comprised an enzyme protein solution of CYP204A1 or CYPSS that had been conserved at each temperature, 64 μg/ml spinach-derived ferredoxin, 0.1 U/ml spinach-derived ferredoxin reductase protein, 3 U/ml glucose dehydrogenase, 60 mM glucose, 50 μg/ml 4-cholesten-3-one, 2 mM NADH, and 2 mM NADPH. The reaction was initiated by addition of NADH and NADPH, and it was carried out at 200 rpm at 15° C. for 4 hours. Thereafter, 1.5 ml of ethyl acetate was added to the reaction solution to terminate the reaction, and extraction was carried out. Then, extraction was carried out again with 0.75 ml of ethyl acetate. The obtained ethyl acetate phase was solidified using an evaporator, and the residue was then dissolved in 200 μl of methanol. Thereafter, progesterone was detected by HPLC analysis. The enzyme protein activity for conversion of 4-cholesten-3-one to progesterone at 0 hour and 140 hours is shown in Table 3. The results demonstrated that, in both cases of CYP204A1 and CYPSS, stability increased as the conservation temperature decreased. In particular, when the cell-free extract was conversed at 20° C. for 140 hours, CYP204A1 maintained 47% of its activity and CYPSS maintained 39% of its activity.

TABLE 3

Activity (μg/ml)

| | | | |
|---|---|---|---|
| Temperature | 0 hr | 140 hr | Relative activity (%) |

CYP204A1

| | | | |
|---|---|---|---|
| 6° C. | 5.3 | 4.5 | 85 |
| 10° C. | 5.3 | 3.9 | 74 |
| 20° C. | 5.3 | 2.5 | 47 |
| 30° C. | 5.3 | 0.5 | 9 |
| 37° C. | 5.3 | 0 | 0 |
| 45° C. | 5.3 | 0 | 0 |

CYPSS

| | | | |
|---|---|---|---|
| 6° C. | 14.4 | 11.6 | 81 |
| 10° C. | 14.4 | 10.5 | 73 |
| 20° C. | 14.4 | 5.6 | 39 |
| 30° C. | 14.4 | 1 | 7 |
| 37° C. | 14.4 | 0 | 0 |
| 45° C. | 14.4 | 0 | 0 |

### (9) Kinetics Analysis of CYP204A1 and CYPSS

Using the cell-free extract prepared in (3) above, conversion of 4-cholesten-3-one was carried out at substrate concentration of 0.1 to 500 μM, and the relationship between the substrate concentration and the enzyme protein activity was examined. The reaction solution was prepared, such that 1 ml of the buffer comprised 220 pmol CYP204A1 or 250 pmol CYPSS, 96 μg/ml spinach-derived ferredoxin, 0.1 U/ml spinach-derived ferredoxin reductase protein, 3 U/ml glucose dehydrogenase, 60 mM glucose, 2 mM NADH, and 2 mM NADPH. Tris-HCl was added to the reaction solution so as to adjust it to pH 7.5. 20 μl of 4-cholesten-3-one, which had been dissolved in DMSO to a final concentration of 0.1, 0.5, 1, 2, 5, 10, 20, 50, 100 or 500 μM, was added. The reaction was initiated by addition of NADH and NADPH, and it was carried out at 200 rpm at 15° C. for 60 minutes. Thereafter, 1.5 ml of ethyl acetate was added to the reaction solution to terminate the reaction, and extraction was carried out. Then, extraction was carried out again with 0.75 ml of ethyl acetate. The obtained ethyl acetate phase was solidified using evaporator, and the residue was then dissolved in 200 μl of methanol. Thereafter, progesterone was detected by HPLC analysis. The relationship between the substrate concentration and the enzyme protein activity, and kinetics parameters calculated by production of Lineweaver-Burk plot, is shown in Table 5. The results demonstrated that CYP204A1 and CYPSS had similar Km values (1.0 and 1.1 μM), and that their Vmax were 47.4 and 78.7 mmol/min/mol, respectively, thereby showing that the Vmax value of CYPSS was approximately 1.7 times higher than that of CYP204A1. In addition, the comparison of these results demonstrated that CYPSS hardly undergoes substrate inhibition.

TABLE 4
Kinetics

| | |
|---|---|
| | Specific activity |
| 4Cholesten-3-on [symbol] | (mmol/min/mol) |

| concn. (μM) | CYP204A1 | | CYPSS | |
|---|---|---|---|---|
| 0.1 | 2.2 | | 1.9 | |
| 0.5 | 15.3 | | 20.0 | |
| 1 | 25.2 | | 36.5 | |
| 2 | 32.2 | | 49.5 | |
| 5 | 36.1 | | 70.4 | |
| 10 | 43.1 | 100 | 67.2 | 100 |
| 20 | 42.7 | | 72.4 | |
| 50 | 33.5 | | 71.2 | |
| 100 | 28.7 | 66.6 | 60.5 | 90.0 |
| 500 | 20.1 | | 33.6 | |

[symbol]

indicates data missing or illegible when filed

TABLE 5
Kinetics parameters

| | Vmax | Km |
|---|---|---|
| CYP204A1 | 47.4 (1.00) | 1.0 |
| CYPSS | 78.7 (1.66) | 1.1 |
| | mmol/min/mol | μM |

( ): Relative activity

The summary of the above-described results is shown below. The optimum pH: pH 7.5 to 8.0 (in both cases of CYP204A1 and CYPSS); the optimum temperature for action: 15° C. to 20° C. (in both cases of CYP204A1 and CYPSS); pH stability: stable around the neutral range, and most stable at KPB pH 7.6 (in both cases of CYP204A1 and CYPSS); thermostability: 30% or more of enzyme protein activity that was retained after conversion at 20° C. for 140 hours (in both cases of CYP204A1 and CYPSS); kinetic analysis: Vmax of CYPSS that was approximately 1.7 times higher than that of CYP204A1, and Km value of CYPSS that was almost equivalent to that of CYP204A1 (1 μM). Moreover, regarding substrate inhibition, it was found that 4-cholesten-3-one causes substrate inhibition, and that substrate inhibition of CYPSS was lower than that of CYP204A1. Furthermore, in comparison with the enzyme protein activity of CYPSS or CYP204A1 when the concentration of 4-cholesten-3-one was 10 μM, the enzyme protein activity when it was 100 μg/ml was 90% or 67%. That is to say, when the substrate concentration was increased from 10 μM to 100 μM, the decrease rate of the enzyme protein activity was 10% or 33%.

### Example 6

### Conversion Reaction of 4-cholesten-3-one by Strain BP215 (Preparation of Progesterone)

The Escherichia coli expression strain BP215 was inoculated into 2 ml of an M9 mix medium (Na2HPO4: 0.68%, KH2PO4: 0.3%, NaCl: 0.05%, NH4C;: 0.01%, Casamino acid: 1%, D-Glucose: 0.4%, CaCl2: 0.1 mM, MgCl2: 1 mM, Thiamine: 0.002%, FeSO4: 0.1 mM, Carbenicillin: 0.005%), and it was then subjected to shaking culture at 30° C. for 17 hours, so as to obtain pre-culture solution. 0.25 ml of this pre-culture solution was inoculated into 25 ml of the main culture medium (Na2HPO4: 0.68%, KH2PO4: 0.3%, NaCl: 0.05%, NH4Cl: 0.01%, Casamino acid: 1%, CaCl2: 0.1 mM, MgCl2: 1 mM, Thiamine: 0.002%, FeSO4: 0.1 mM, Carbenicillin: 0.005%, Overnight Express™ solution: 7%, 5-Aminolevlic acid: 0.008%) using Overnight Express™ Autoinduction System (Novagen Cat. No. 71300-4), and it was then subjected to shaking culture at 25° C. for 24 hours. 5 ml of the obtained main culture solution was centrifuged (3500 rpm, 10 minutes) to obtain cell mass, and 1 ml of CV2 buffer (50 mM (pH) potassium phosphate buffer containing glycerol: 2%, carbenicillin: 0.005%, and isopropyl β-D-1-thiogalactopyranoside: 0.1 mM) was added to the obtained cell mass. Thereafter, 0.01 ml of the methanol solution of 1% 4-cholesten-3-one and 0.02 ml of an aqueous solution of 25% methyl-β-cyclodextrin were added to the mixed solution, and the obtained mixture was then shaken at 28° C. for 5 hours for the reaction. After completion of the reaction, 4 ml of methanol was added to the reaction solution, and they were then fully mixed. The obtained mixture was centrifuged, and the upper layer was then analyzed by HPLC. The peak of progesterone was detected at retention time of 2.6 minutes, and thus, the progesterone preparation, the retention time, and the MS spectrum (m/z=315 M+1) were matched. The accumulated amount was found to be 78.0 mg/l (conversion rate: 78%). On the other hand, 4-cholesten-3-one (retention time: 8.1 minutes) was not detected.

Analysis conditions: Using Chromolith speed rod RP18e (50×4.6 mm) column, 50%→100% CH3CN gradient (0 to 3 min) and 100% CH3CN (3 to 8 min) were used as a mobile phase, and detection was carried out at a flow rate of 2 ml/min using CAD™ (Charged Aerosol Detection) and MS.

### Example 7

### Conversion Reaction of Cholesterol by Strain BP215

Using cholesterol as substrate, the same reaction as that of Example 6 was carried out, and treatment and analysis were then carried out. As a result, the peak of pregnenolone was detected at retention time of 2.5 minutes, and thus, the pregnenolone preparation, the retention time, and the MS spectrum (m/z=317 M+1) were matched. The accumulated amount was found to be 68.2 mg/l (conversion rate: 68.2%). On the other hand, 3.7% of cholesterol (retention time: 8.5 minutes) remained.

### Example 8

### Conversion Reaction of 7-Dehydrocholesterol and Ergosterol by Strain BP215

Using 7-dehydrocholesterol as substrate, the same reaction as that of Example 6 was carried out, and treatment and analysis were then carried out. As a result, the peak of 7-dehydropregnenolone (m/z=315 M+1) was detected at retention time of 2.1 minutes. The accumulated amount was found to be 34.3 mg/l (conversion rate: 34.3%). On the other hand, 54% of 7-dehydrocholesterol (retention time: 7.5 minutes) remained.

Using ergosterol as substrate, the same reaction as that of Example 6 was carried out, and treatment and analysis were then carried out. As a result, the peak of 7-dehydropregnenolone was detected at a retention time of 2.1 minutes. The accumulated amount was found to be 2.8 mg/l (conversion rate: 2.8%).

### Example 9

### Conversion Reaction of Various Types of Substrates by Strain BP215

As substrate, β-sitosterol, stigmasterol, campesterol or desmosterol was used. Each substrate was subjected to the same reaction as that of Example 6, separately, and the treatment and the analysis were then carried out. As a result, the peak of pregnenolone was detected at the retention time of 2.5 minutes, and thus, the pregnenolone preparation, the retention time, and the MS spectrum (m/z=317 M+1) were matched. The accumulated amounts were found to be 40.9 mg/l (conversion rate: 40.9%), 5.8 mg/l (conversion rate: 5.8%), 27.9 mg/l (conversion rate: 27.9%), and 71.7 mg/l (conversion rate: 71.7%), respectively. On the other hand, the added substrate (the retention time was 10.1, 9.3, 9.2, and 7.2 minutes, respectively) remained at 55.9%, 70.8%, 25.5%, and 21.3%, respectively. It is to be noted that since the product generated from β-sitosterol was in an extremely small amount, the reaction solution was extracted with ethyl acetate, was then concentrated by a factor of 5, and was then subjected to the analysis.

### Example 10

### Chemical synthesis of (20R,22R)-20,22-dihydroxy-4-cholest-4-en-3-one and (20R,22S)-20,22-dihydroxycholest-4-en-3-one

The synthesis of two stereoisomers of 20,22-dihydroxycholest-4-en-3-one was carried out by going through 20,22-dihydroxycholesterol according to the synthesis method described in the known publication, and then applying an ordinary oxidation reaction.

### (A) Synthesis of 20,22-dihydroxycholesterol (10-A)

The chemical synthesis of the two isomers [a (20R,22R) form and a (20R,225) form] of the compound (10-A) is described in Steroids (2004), 69, 483/B. Watanabe et al. Thus, the compound (10-A) was synthesized in accordance with the experimental methods described in the above-mentioned publication.

The essence of the synthesis is that pregnenolone was used as a starting material, the group at position 3 thereof was protected by t-butyldimethylsilyl chloride, and 4-methyl-1-pentine was added thereto to obtain (20R)-3β-(t-butyldimethylsilyloxy)cholest-5-en-22-yn-20-ol. This compound was reduced to cis-olefin, using Lindlar catalyst, so as to obtain (20R,22Z)-3β-(t-butyldimethylsilyloxy)cholest-5,22-dien-20-ol. This compound was converted to 22,23-epoxide (an isomeric mixture) by VO(acac)₂/TBHP, and it was kept as an isomeric mixture without being separated. It was reduced by LiAlH4, and was converted to 20,22-dihydroxy form. Thereafter, the group at position 3 thereof was deprotected by TBAF, so as to obtain (20R,22RS)-20,22-dihydroxycholesterol (10-A).

### (B) Synthesis of 20,22-dihydroxycholest-4-en-3-one (10-B)

With reference to the method described in the above-mentioned publication, (20R,22RS)-20,22-O-isopropylidenedioxycholesterol was first obtained from the compound (10-A) [wherein 22-dimethoxypropane was allowed to act thereon in the presence of PPTS]. The obtained compound was converted to the 4-en-3-one form by Oppenauer oxidation according to an ordinary method. The experimental procedures will be specifically described below.

(20R,22RS)-20,22-O-isopropylidenedioxycholesterol (3.0 g) was dissolved in 100 mL of toluene in Dean-Stark apparatus that had been sufficiently subjected to nitrogen substitution, and 1-methyl-4-piperidone (8.1 mL, 10 eq) was then added to the solution Thereafter, the reaction temperature was set at 130° C. to 140° C., Al(OiPr)₃ (2.7 g, 2 eq) was then added to the reaction solution, and the obtained mixture was then stirred for 4 hours. After completion of the reaction, the reaction solution was diluted with diethyl ether, was then subjected to ordinary treatments, and was then purified by silica gel chromatography [hexane/ethyl acetate=8/1 to 5/1], so as to obtain (20R,22RS)-20,22-O-isopropylidenedioxycholest-4-en-3-one (2.9 g, y 97%).

Finally, (20R,22RS)-20,22-O-isopropylidenedioxycholest-4-en-3-one was converted to the compound (10-B) by deprotecting acetonide, and further, two stereoisomers could be obtained, separately. Specific experimental procedures will be described below.

(20R,22RS)-20,22-O-isopropylidenedioxycholest-4-en-3-one (2.9 g) was stirred at room temperature in THF (20 mL)+methanol (20 mL). In the mean time, 2 N hydrochloric acid (1.0 mL) and perchloric acid (0.8 mL) were added thereto, and the reaction was then carried out for 60 hours. Then, the reaction solution was diluted with chloroform and was then subjected to ordinary treatments. Thereafter, the reaction solution was purified by silica gel chromatography [hexane/ethyl acetate=7/1 to 2/1], so that a deprotected product (1.48 g, y 47%) was separated from an unreacted raw material (1.24 g), and thus they were obtained, separately. A white solid (1.1 g) was obtained from the deprotected product by recrystallization from hexane/ethyl acetate.

1H-NMR (400 MHz, CDCl₃) δ: 0.90 (d, J=6.6 Hz, 26-H₃), 0.90 (d, J=6.8 Hz, 27-H₃), 0.91 (s, 18-H₃), 1.19 (s, 19-H₃), 1.26 (s, 21-H₃), 3.24 (dd, J=9.4 Hz, 5.4 Hz, 22-H), 5.73 (s, 4-H)→(20R,22S)-20,22-dihydroxycholest-4-en-3-one (10-B) [non-natural isomer, which is a novel compound obtained by the present invention]

The recovered unreacted raw material (590 mg) was stirred at room temperature in THF (15 mL)+methanol (15 mL). In the meantime, perchloric acid (1.4 mL) was added thereto, and the reaction was then carried out for 10 hours. Then, the reaction solution was diluted with chloroform and was then subjected to ordinary treatments. Thereafter, the reaction solution was purified by silica gel chromatography [hexane/ethyl acetate=7/1 to 2/1], so as to obtain a deprotected product (217 mg, y 40%).

1H-NMR (500 MHz, CDCl₃) δ: 0.89 (d, J=6.3 Hz, 26-H₃), 0.91 (d, J=6.4 Hz, 27-H₃), 0.93 (s, 18-H₃), 1.19 (s, 19-H₃), 1.21 (s, 21-H₃), 3.38 (d, J=8.4 Hz, 22-H), 5.73 (s, 4-H)→(20R,22R)-20,22-dihydroxycholest-4-en-3-one (10-B) [natural isomer; (citation) Helv. Chim. Acta, (2006), 89, 813/W. Zhang et al.]

### Example 11

### Conversion Reaction of Various Types of Substrates by Strain BP215

As substrates, (20S)-20-hydroxycholest-4-en-3-one, (22R)-22-hydroxycholest-4-en-3-one, (20R,22R)-20,22-dihydroxycholest-4-en-3-one, and (20R,22S)-20,22-dihydroxycholest-4-en-3-one were used. Each substrate was subjected to the same reaction as that of Example 6, separately, and the generated product was then analyzed by HPLC. As a result, generation of progesterone was confirmed. The conversion rates were found to be 41.0%, 42.3%, 38.1% and 69.8%, respectively. On the other hand, the added substrates (their retention times were 4.9, 4.4, 3.8, and 3.5 minutes, respectively) remained at 4.8%, 2.7%, 0.6%, and 0.6%, respectively.

Moreover, as substrates, (20S)-20-hydroxy-cholesterol, (22S)-22-hydroxy-cholesterol, (22R)-22-hydroxy-cholesterol, and (20R,22R)-20,22-dihydroxy-cholesterol were used. Each substrate was subjected to the same reaction as that of Example 6, separately, and treatment and analysis were then carried out. As a result, generation of pregnenolone as a side-chain cleavage body corresponding to each substrate was confirmed. The accumulated amounts were found to be 89.2 mg/L (conversion rate: 89.2%), 37.5 mg/L (conversion rate: 37.5%), 80.0 mg/L (conversion rate: 80%), and 53.0 mg/L (conversion rate: 53.0%), respectively. On the other hand, the added substrates remained at 0%, 23.5%, 14.8%, and 0%, respectively.

### Example 12

### Conversion Reaction of Various Types of Substrates by Strain BP172

As substrates, 4-cholesten-3-one, cholesterol, 7-dehydrocholesterol, ergosterol, 13-sitosterol, stigmasterol, campesterol, desmosterol, (20S)-20-hydroxycholest-4-en-3-one, (22R)-22-hydroxycholest-4-en-3-one, (20R,22R)-20,22-dihydroxycholest-4-en-3-one, and (20R,22S)-20,22-dihydroxycholest-4-en-3-one were used. Each substrate was subjected to the same reaction as that of Example 6, separately, and the generated product was then analyzed by HPLC. As a result, generation of progesterone, pregnenolone or 7-dehydropregnenolone as a side-chain cleavage body corresponding to each substrate was confirmed. The conversion rates were found to be 84.0%, 71.2%, 28.5%, 22%, 47.2%, 6.5%, 31.3%, 66.1%, 63.9%, 80.9%, 61.3% and 41.7%, respectively.

Moreover, as substrates, (20S)-20-hydroxy-cholesterol, (22S)-22-hydroxy-cholesterol, (22R)-22-hydroxy-cholesterol, and (20R,22R)-20,22-dihydroxy-cholesterol were used. Each substrate was subjected to the same reaction as that of Example 6, separately, and treatment and analysis were then carried out. As a result, generation of pregnenolone as a side-chain cleavage body corresponding to each substrate was confirmed. The accumulated amounts were found to be 68.2 mg/L (conversion rate: 68.2%), 12.9 mg/L (conversion rate: 12.9%), 69.5 mg/L (conversion rate: 69.5%), and 51.6 mg/L (conversion rate: 51.6%), respectively. On the other hand, the added substrates remained at 25.2%, 60.1%, 42.8% and 0%, respectively.

### Reference Example 1

### Production of CYP11A-Expressing Escherichia coli

A commercially available human testis cDNA (Origene Technologies, Inc.) was used as a template, and a CYP11A1 gene was amplified by PCR using a primer CYP11A-1F (SEQ ID NO: 19) and a primer CYP11A-2R (SEQ ID NO: 20), and also using KOD plus polymerase (TOYOBO Co., Ltd.). The temperature conditions applied during the PCR were 94° C./3 min, 30 cycles of (94° C./30 sec, 55° C./60 sec, and 72° C./90sec), and 72° C./5 min.

A 1.45-kb CYP11A1 gene fragment amplified as a result of the above-described reaction was purified using QIAquick PCR purification Kit (manufactured by QIAGEN), and it was then digested with Nde I and Spe I. Thereafter, the digest was ligated to Escherichia coli expression vector pT7NS-camAB, using T4 DNA ligase, and Escherichia coli DH5α was then transformed with the obtained expression vector, so as to construct a plasmid pCYP11A-camAB. Subsequently, using T4 DNA ligase, the previously prepared bovine-derived adrenodoxin reductase protein-adrenodoxin gene (adr-adx) fragment was inserted into and ligated to each of the Spe I and Bam HI sites of the above-described plasmid, and Escherichia coli DH5a was then transformed with the obtained plasmid, so as to construct plasmid pCYP11A-ARX. Thereafter, Escherichia coli BL21(DE3) was transformed with the thus constructed plasmid, and the obtained cell strain was named as BL21CYP11. The used adx-adr fragment was amplified by PCR with a plasmid pKARX (Sawada et. al. Eur. J. Biochem. 265, 950-956,1999) as a template, using primer bAdxR-1F (SEQ ID NO: 21) and primer bAdx-2R (SEQ ID NO: 22), and also using KOD plus polymerase (manufactured by TOYOBO Co., Ltd.). The temperature condition applied during the PCR were 94° C./3 min, 25 cycles of (94° C./30 sec, 60° C./30 sec, and 72° C./120sec), and 72° C./5 min.

A 1.85-kb adr-adx gene fragment amplified as a result of the above-described reaction was purified using QIAquick PCR purification Kit (manufactured by QIAGEN), and it was then digested with Spe I and Fba I. Thereafter, the digest was used to construct the above-described pCYP11A-ARX.

### Comparative Example

### Conversion Reaction of Various Types of Substrates by CYP11A-Expressing Escherichia coli

As substrate, 4-cholesten-3-one (4Chol), cholesterol (Chol), 7-dehydrocholesterol (7-DHC), ergosterol (Ergo), β-sitosterol (Sito), stigmasterol (Stigma), campesterol (Cam), desmosterol (Des), (20S)-20-hydroxycholest-4-en-3-one (20-OH), (22R)-22-hydroxycholest-4-en-3-one (22-OH), (20R,22R)-20,22-dihydroxycholest-4-en-3-one (R-diol), (20R,22S)-20,22-dihydroxycholest-4-en-3-one (S-diol), or lanosterol (Lano) was used. Each substrate was subjected to the same reaction as that of Example 6, separately, and the reaction solution was then subjected to liquid separation using 1 ml of ethyl acetate. The reaction product and the substrate were extracted, and were then analyzed by HPLC. As a result, generation of progesterone, pregnenolone or 7-dehydropregnenolone as the side-chain cleavage body corresponding to each substrate was confirmed. The conversion rates were found to be 0%, 2.6%, 0.7%, 0%, 0.8%, 0%, 1.0%, 1.2%, 0%, 0.4%, 4.3% and 0%, respectively. On the other hand, the added substrates remained at 100%, 99%, 99.2%, 100%, 99.7%, 100%, 99.1%, 99.6%, 100%, 99.7%, 95% and 100%, respectively.

Moreover, as substrate, (20S)-20-hydroxy-cholesterol, (22S)-22-hydroxy-cholesterol, (22R)-22-hydroxy-cholesterol, or (20R,22R)-20,22-dihydroxy-cholesterol was used. Each substrate was subjected to the same reaction as that of Example 6, separately, and the treatment and the analysis was then carried out. As a result, generation of pregnenolone as the side-chain cleavage body corresponding to each substrate was confirmed. The accumulated amount was found to be 16.3 mg/L (conversion rate: 16.3%), 6.1 mg/L (conversion rate: 6.1%), 28.0 mg/L (conversion rate: 28.0%), and 32.3 mg/L (conversion rate: 32.3%), respectively. On the other hand, the added substrates remained at 98.8%, 92.6%, 78.6% and 51.6%, respectively.

A summary of the conversion reaction rate to individual substrate is shown in the following Table 6.

TABLE 6
Conversion rate (%)

| | 4Chol | S-diol | R-diol | 20-OH | 22OH | Chol | Stigma | Lano | Sito | Cam | Des | 7-DHC | Ergo |
|---|---|---|---|---|---|---|---|---|---|---|---|---|---|
| CYP11 | 0 | 0 | 4.3 | 0 | 0.4 | 2.6 | 0 | 0 | 0.8 | 1 | 1.2 | 0.7 | 0 |
| BP172 | 84 | 41.7 | 61.3 | 63.9 | 80.9 | 71.2 | 6.5 | 0 | 47.2 | 31.3 | 66.1 | 28.5 | 2.2 |
| BP215 | 78 | 69.8 | 38.1 | 41 | 42.3 | 68.2 | 5.8 | 0 | 40.9 | 27.9 | 71.7 | 34.3 | 2.8 |

### Example 13

### SDS-PAGE

BL21CYP11A, BP172 and BP215 were each inoculated into 2 ml of TB medium comprising 50 μg/ml (the final concentration) ampicillin, and they were then subjected to shaking culture at 30° C. at 220 rpm for 16 hours. 250 μl of the obtained pre-culture was added to 25 ml of TB medium used for main culture (ampicillin 50 μg/ml <final concentration>, 5-aminolevulinic acid 40 μg/ml <final concentration>, and inducer: Overnight Express Autoinduction System 1 <manufactured by Merck>), and it was then subjected to the shaking culture at 25° C. for 24 hours. The cell mass recovered from the culture solution by centrifugation was suspended in 5 ml of buffer (50 mM KPB, 2% glycerol [pH 7.4]) so as to prepare the cell suspension. To 2 ml of this cell suspension, 66.7 μl of ×10 Bug Buster (manufactured by Novagen), 0.67 μl of Benzonase (manufactured by Novagen), and 40 mg/ml lysozyme were added, and the obtained mixture was then shaken at 30° C. for 20 minutes so that the cells were lysed. This cell lysis solution was defined as the total protein solution. Moreover, the cell lysis solution was centrifuged, and the obtained supernatant was defined as the soluble fraction protein solution. The insoluble fraction protein was prepared by centrifuging the cell lysis solution, completely removing the centrifuge supernatant, washing the residue with buffer 3 times, and then re-suspending the resultant in the buffer. These total protein solution, soluble fraction protein solution and insoluble fraction protein solution were each mixed with 20 μl of the SDS-PAGE sample buffer (manufactured by BIO-RAD), so as to prepare samples to be used for SDS-PAGE electrophoresis. The SDS-PAGE electrophoresis sample was heated at 95° C. for 5 minutes, and was then electrophoresed (300 V, 30 mA, 60 minutes) on polyacrylamide gel (manufactured by Daiichi Kagaku Yakuhin Kabushiki Kaisha). After completion of the electrophoresis, the polyacrylamide gel was transferred onto a tray, and an appropriate amount of staining-decolorating solution (manufactured by Takara Bio INC.) was then added thereto, so as to carry out staining and decoloration. The results are shown in FIG. 1.

### INDUSTRIAL APPLICABILITY

Using the novel enzyme protein of the present invention, it became possible to efficiently produce pregnenolone and the like and hydrocortisone, which are compounds industrially useful as medicaments and pharmaceutical intermediates.

### BRIEF DESCRIPTION OF THE DRAWINGS

FIG. 1 is a view showing the results of SDS-PAGE using BL21CYP11A, BP172 and BP215.

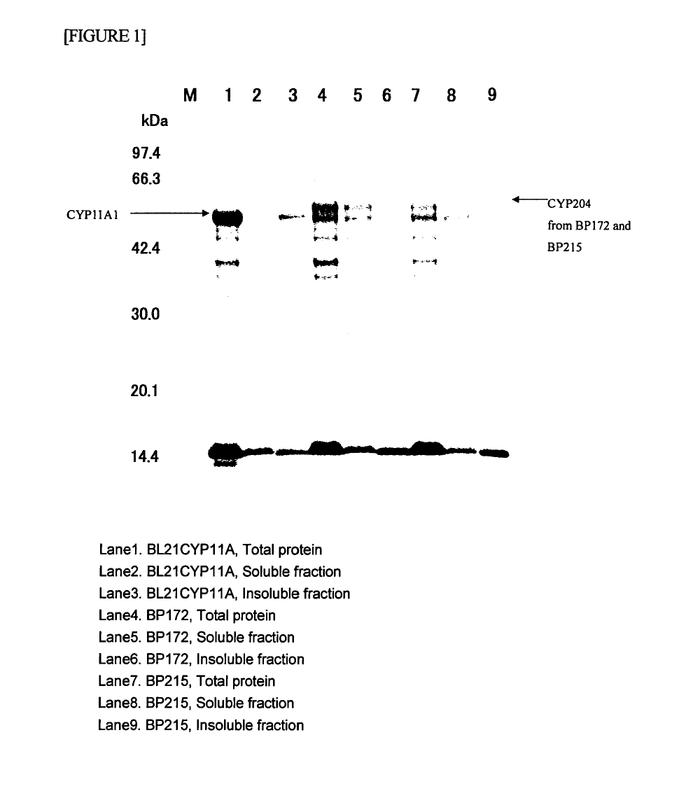

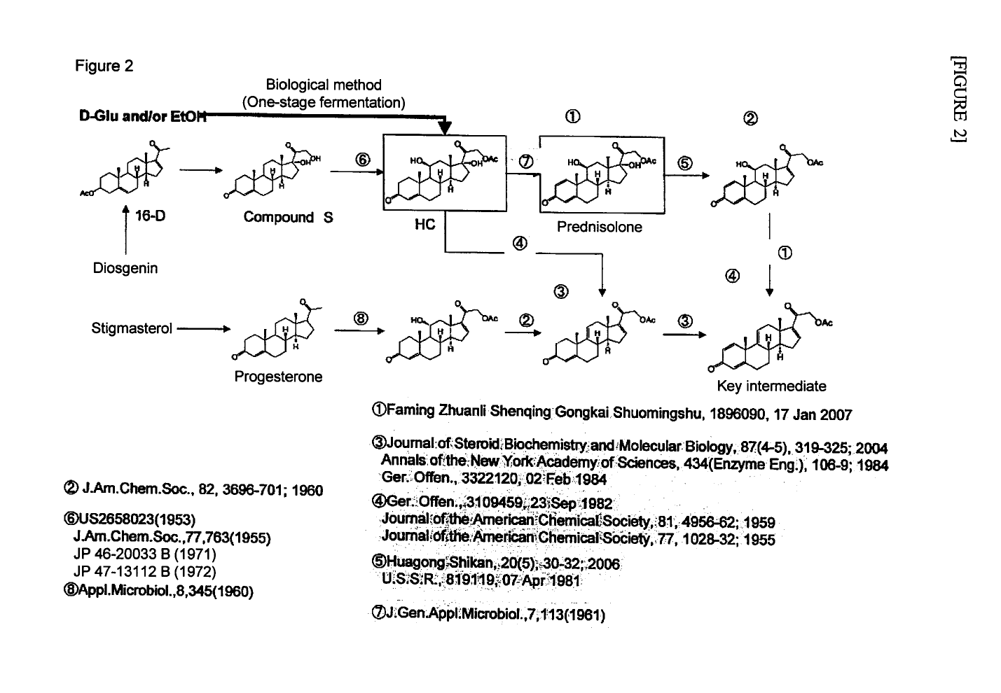

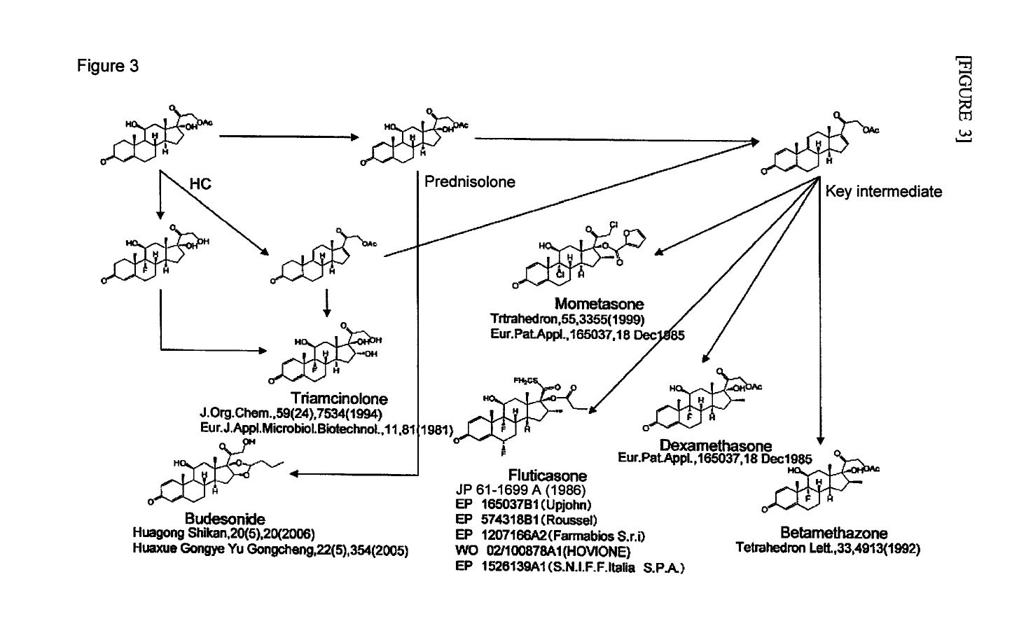

FIG. 2 is a view showing derivatives of hydrocorticoid and a synthetic method thereof.
1. Faming Zhuanli Shenqing Gongkai Shuomingshu, 1896090, 17 Jan. 2007
2. J. Am. Chem. Soc., 82, 3696-701; 1960
3. Journal of Steroid Biochemistry and Molecular Biology, 87(4-5), 319-325; 2004; and Annals of the New York Academy of Sciences, 434 (Enzyme Eng.), 106-9; 1984 Ger. Offen., 3322120, 2 Feb. 1984
4. Ger. Offen., 3109459, 23 Sep. 1982; Journal of the American Chemical Society, 81, 4956-62; 1959; and Journal of the American Chemical Society, 77, 1028-32; 1955
5. Huagong Shikan, 20(5), 30-32; 2006, U.S.S.R., 819119, 7 Apr. 1981
6. U.S. Pat. No. 2,658,023 (1953); J. Am. Chem. Soc., 77, 763 (1955); JP Patent Publication (Kokoku) No. 46-20033 B (1971); JP Patent Publication (Kokoku) No. 47-13112 B (1972)
7. J. Gen. Appl. Microbiol., 7, 113 (1961)
8. Appl. Microbiol., 8, 345 (1960)

FIG. 3 is a view showing derivatives of hydrocorticoid and a synthetic method thereof.
Mometasone: Tetrahedron, 55, 3355 (1999); and Eur. Pat. Appl., 165037, 18 Dec. 1985 Triamcinolone; J. Org. Chem., 59(24), 7534 (1994); and Eur. J. Appl. Microbiol. Biotechnol., 11, 81 (1981)
Dexamethasone; Eur. Pat. Appl., 165037, 18 Dec. 1985
Budesonide; Huagong Shikan, 20(5), 20 (2006); and Huaxue Gongye Yu Gongcheng, 22(5), 354 (2005)
Fluticasone; JP Patent Publication (Kokai) No. 61-1699 A (1986); EP 165037 B1; EP 574318 B1; EP 1207166 A2; WO02/100878 A1; and EP 1526139 A1
Betamethazone: Tetrahedron Lett., 33, 4913 (1992)

1. A protein selected from the group consisting of:
(a) a protein consisting of the amino acid sequence shown in SEQ ID NO: 2;
(b) a protein consisting of an amino acid sequence comprising a deletion, substitution and/or addition of at least one amino acid with respect to the amino acid sequence shown in SEQ ID NO: 2, and having an activity (b) of cleaving the bond between positions 20 and 22 of a sterol side chain, wherein the maximum velocity (Vmax) that is a reaction rate parameter of the activity (b) is 50 mmol/min/mol or more; and
(c) a protein consisting of an amino acid sequence having homology of 95% or more with the amino acid sequence shown in SEQ ID NO: 2, and having an activity (c) of cleaving the bond between positions 20 and 22 of a sterol side chain, wherein the maximum velocity (Vmax) that is a reaction rate parameter of the activity (c) is 50 mmol/min/mol or more.

2. A sterol side chain cleavage enzyme having the following physicochemical properties:
(1) an activity, wherein the enzyme acts on a sterol represented by formula (I) and cleaves the carbon-carbon bond between positions 20 and 22 of a sterol side chain portion to generate a compound represented by formula (II);
(2) a substrate specificity, wherein when at least one microorganism that produces the enzyme is allowed to react with an aqueous solution comprising 100 μg/ml 4-cholesten-3-one or cholesterol at 28° C. for 5 hours, a conversion reaction rate from 4-cholesten-3-one to progesterone is 10% or more, and a conversion rate from cholesterol to pregnenolone is 10% or more;
(3) an optimum pH of 7.5 to 8.0;
(4) an optimum temperature for the activity is 15° C. to 20° C.;
(5) a thermostability, wherein after preservation at 20° C. for 140 hours, 30% or more of enzyme activity is maintained; and
(6) a molecular weight, wherein the putative molecular weight is 53 to 54 KDa based on the amino acid sequence, and it is measured to be 50 to 56 KDa by SDS electrophoresis, 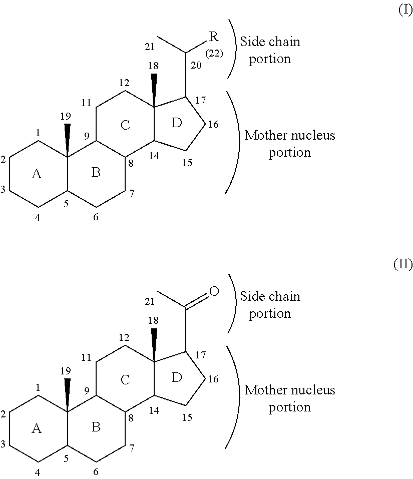
wherein, in the formula (I),
a mother nucleus portion (I) consists of the A, B, C and D rings of a steroid, wherein the mother nucleus portion (I) has at least one carbon-carbon unsaturated bond at a zero site or at one or more sites of the positions 1 to 17 of the mother nucleus portion (I), except for positions 10 and 13, and
at least one first carbon at the zero site, or at one or more sites of the positions 1 to 19 of the mother nucleus portion (I), except for positions 10 and 13, is independently substituted with
a group of the formula —OX, wherein:
X═H;
X═COR₁ wherein R₁ is a hydrogen atom, or an alkyl group, alkenyl group, alkynyl group or aromatic hydrocarbon comprising 10 or less carbon atoms;
X═R₂ wherein R₂ is an alkyl group, alkenyl group or alkynyl group comprising 10 or less carbon atoms, and optionally substituted with an oxygen atom;
X═SO₃M wherein M is a hydrogen atom, an alkaline metal, or an alkaline-earth metal;
—OX is an O-glycosyl group, wherein X is a carbon at position 1 of a sugar; or
—OX is an epoxy group, wherein X is a carbon atom adjacent to the at least one first carbon, or
a keto group of the formula ═O, and
in a side chain portion (I), R is:
a linear alkyl group, an alkenyl group or alkynyl group comprising 10 or less carbon atoms, and optionally further comprising a cyclic portion; or
a branched alkyl group, alkenyl group or alkynyl group comprising 10 or less carbon atoms, and optionally further comprising a cyclic portion,
the carbons at positions 20 and 21, and at the zero site or at one or more sites in the R, are independently substituted with
a group represented by the formula —OY, wherein:
Y═H;
Y═COR₃ wherein R₃ is a hydrogen atom, or an alkyl group, alkenyl group, alkynyl group or aromatic hydrocarbon comprising 10 or less carbon atoms;
Y═R₄ wherein R₄ is an alkyl group, alkenyl group or alkynyl group comprising 10 or less carbon atoms, and optionally substituted with an oxygen atom;
Y═SO₃M wherein M is a hydrogen atom, an alkaline metal or an alkaline-earth metal;
—OY is an O-glycosyl group, wherein Y is a carbon at position 1 of a sugar; or
—OY is an epoxy group, wherein Y is a carbon atom adjacent to the carbons, or
a keto group represented by the formula ═O, and
wherein in the formula (II),
a mother nucleus portion (II) is defined the same as the mother nucleus portion (I), and
in a side chain portion (II), the carbon at position 21 is substituted with
zero or one group represented by the formula —OY, wherein:
Y═H;
Y═COR₃ wherein R₃ is a hydrogen atom, or an alkyl group, alkenyl group, alkynyl group or aromatic hydrocarbon containing 10 or less carbon atoms;
Y═R₄ wherein R₄ is an alkyl group, alkenyl group or alkynyl group comprising 10 or less carbon atoms and optionally substituted with an oxygen atom;
Y═SO₃M wherein M is a hydrogen atom, an alkaline metal, or an alkaline-earth metal; or
—OY is an O-glycosyl group, wherein Y is the carbon at position 1 of a sugar, or
zero or one keto group of the formula ═O.

3. DNA encoding the protein of claim 1.

4. DNA selected the group consisting of:
(a) DNA having the nucleotide sequence shown in SEQ ID NO: 1;
(b) DNA having a nucleotide sequence comprising a deletion, substitution and/or addition of at least one nucleotide with respect to the nucleotide sequence shown in SEQ ID NO: 1, and encoding a protein with an activity (b) of cleaving the bond between positions 20 and 22 of a sterol side chain, wherein the maximum velocity (Vmax) that is a reaction rate parameter of the activity (b) is 50 mmol/min/mol or more; and
(c) DNA having a nucleotide sequence capable of hybridizing under stringent conditions with the DNA having the nucleotide sequence shown in SEQ ID NO: 1 or a complementary sequence thereof, and encoding a protein with an activity (c) of cleaving the bond between positions 20 and 22 of a sterol side chain, wherein the maximum velocity (Vmax) that is a reaction rate parameter of the activity (c) is 50 mmol/min/mol or more.

5. A fusion protein, wherein
the protein of claim 1,
a ferredoxin protein having electron-transferring activity on the protein, and
a ferredoxin reductase protein having electron-transferring activity on the ferredoxin protein
are allowed to bind to one another, so that they can function as a single protein.

6. A recombinant vector, comprising the DNA of claim 3 and an expression control region capable of expressing a protein encoded by the DNA in a host cell.

7. A transformant formed by introducing the recombinant vector of claim 6 into the host cell.

8. A method for producing a compound represented by formula (II), comprising contacting a sterol represented by formula (I) with a mixture of the protein of claim 1, a ferredoxin protein having electron-transferring activity on the protein, and a ferredoxin reductase protein having electron-transferring activity on the ferredoxin protein.

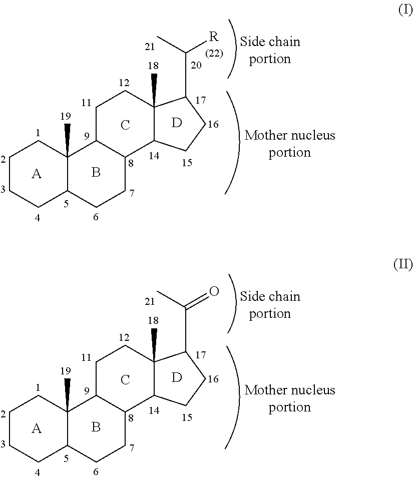

9. A method for producing a compound of formula (II), comprising contacting a sterol represented by formula (I) with a composition selected from the group consisting of:
(a) a mixture of a protein consisting of the amino acid sequence shown in SEQ ID NO: 23, a ferredoxin protein having electron-transferring activity on the protein, and a ferredoxin reductase protein that transfers electrons to the ferredoxin protein;
(b) a mixture of:
a protein (b) consisting of an amino acid sequence comprising a deletion, substitution and/or addition of at least one amino acid with respect to the amino acid sequence shown in SEQ ID NO: 23, and having an activity (b) of cleaving the bond between positions 20 and 22 of a sterol side chain, wherein the maximum velocity (Vmax) that is a reaction rate parameter of the activity (b) is 40 mmol/min/mol or more;
a ferredoxin protein (b) having electron-transferring activity on the protein (b); and
a ferredoxin reductase protein (b) that transfers electrons to the ferredoxin protein (b); and
(c) a fusion protein, wherein:
a protein (c) consisting of the amino acid sequence shown in SEQ ID NO: 23 or a protein consisting of an amino acid sequence comprising a deletion, substitution and/or addition of at least one amino acid with respect to the amino acid sequence shown in SEQ ID NO: 23, and having an activity (c) of cleaving the bond between positions 20 and 22 of a sterol side chain, wherein the maximum velocity (Vmax) that is a reaction rate parameter of the activity (c) is 40 mmol/min/mol or more,
a ferredoxin protein (c) having electron-transferring activity on the protein (c), and
a ferredoxin reductase protein (c) having electron-transferring activity on the ferredoxin protein (c),
are allowed to bind to one another, so that they can function as a single protein, and 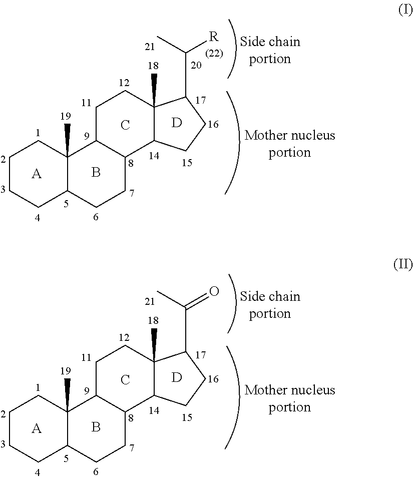
the sterol is represented by formula (I) of claim 2 and the compound is represented by formula (II) of claim 2.

10. The method of claim 8, further comprising contacting the compound of formula (II) with at least one enzyme selected from the group consisting of 3beta-hydroxysteroid dehydrogenase, 3beta-hydroxysteroid:oxygen oxidoreductase, steroid 17α-hydroxylase, steroid 21-hydroxylase and steroid 11β-hydroxylase to generate a hydrocortisone.

11. The method of claim 8, wherein the sterol represented by formula (I) is cholesterol, 4-cholesten-3-one, 7-dehydrocholesterol, ergosterol, β-sitosterol, stigmasterol, campesterol, desmosterol, (20S)-20-hydroxycholest-4-en-3-one, (22R)-22-hydroxycholest-4-en-3-one, (20R,22R)-20,22-dihydroxycholest-4-en-3-one, or (20R,22S)-20,22-dihydroxycholest-4-en-3-one.

12. The method of claim 8, wherein the compound represented by the formula (II) is pregnenolone, progesterone, or 7-dehydropregnenolone.

13. The method of claim 8, wherein the sterol represented by the formula (I) is generated by contacting a raw material selected from the group consisting of glucose, glycerin, methanol, ethanol, saccharose, acetic acid and citric acid 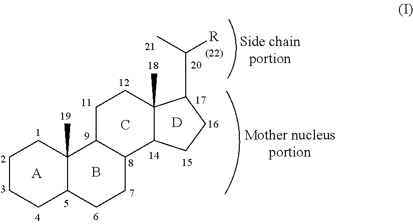
with a yeast having an ability to assimilate the raw material to generate the sterol represented by the formula (I).

14. A method for producing hydrocortisone, comprising culturing a yeast with a raw material in a medium containing the raw material,
wherein
the raw material is selected from the group consisting of glucose, glycerol, methanol, ethanol, sucrose, acetic acid and citric acid,
the yeast has an activity of generating a sterol represented by formula (I) from the raw material, 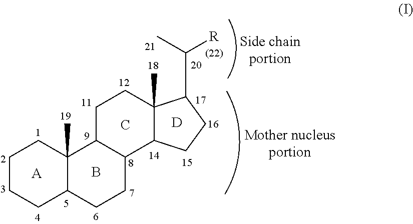
the yeast has an activity of generating at least one enzyme selected from the group consisting of 3beta-hydroxysteroid dehydrogenase, 3beta-hydroxysteroid:oxygen oxidoreductase, steroid 17α-hydroxylase, steroid 21-hydroxylase and steroid 11β-hydroxylase, and
the yeast has any one additional activity selected from the group consisting of:
(A) an activity (A) of generating a mixture of the protein of claim 1, a ferredoxin protein with electron-transferring activity on the protein, and a ferredoxin reductase protein with electron-transferring activity on the ferredoxin protein;
(B) an activity (B) of generating a mixture of a protein (B) consisting of the amino acid sequence shown in SEQ ID NO: 23, a ferredoxin protein having electron-transferring activity on the protein (B), and a ferredoxin reductase protein (B) that transfers electrons to the ferredoxin protein (B);
(C) an activity (C) of generating a mixture of:
a protein (C) consisting of an amino acid sequence comprising deletion, substitution and/or addition of at least one amino acid with respect to the amino acid sequence shown in SEQ ID NO: 23, and having an activity (C1) of cleaving the bond between positions 20 and 22 of a sterol side chain, wherein the maximum velocity (Vmax) that is a reaction rate parameter of the activity (C1) is 40 mmol/min/mol or more;
a ferredoxin protein (C) having electron-transferring activity on the protein (C); and
a ferredoxin reductase protein (C) that transfers electrons to the ferredoxin protein (C);
(D) an activity (D) of generating a fusion protein (D), wherein
a protein (D) consisting of the amino acid sequence shown in SEQ ID NO: 23 or a protein consisting of an amino acid sequence comprising a deletion, substitution and/or addition of at least one amino acid with respect to the amino acid sequence shown in SEQ ID NO: 23, and having an activity (D) of cleaving the bond between positions 20 and 22 of a sterol side chain, wherein the maximum velocity (Vmax) that is a reaction rate parameter of the activity (D) is 40 mmol/min/mol or more,
a ferredoxin protein (D) having electron-transferring activity on the protein (D), and
a ferredoxin reductase protein (D) having electron-transferring activity on the ferredoxin protein (D)
are allowed to bind to one another, so that they can function as a single protein; and
(E) an activity (E) of generating a fusion protein (E), wherein
the protein of claim 1,
the ferredoxin protein having electron-transferring activity on the protein, and
the ferredoxin reductase protein having electron-transferring activity on the ferredoxin protein
are allowed to bind to one another, so that they can function as a single protein.

15. A fusion protein, wherein
the protein of claim 2,
a ferredoxin protein having electron-transferring activity on the protein, and
a ferredoxin reductase protein having electron-transferring activity on the ferredoxin protein
are allowed to bind to one another, so that they can function as a single protein.

16. The method of claim 8, wherein the sterol is represented by the formula (I) of claim 2 and the compound is represented by the formula (II) of claim 2.

17. A method for producing a compound, comprising contacting a sterol with a mixture of the protein of claim 2, a ferredoxin protein with electron-transferring activity on the protein, and a ferredoxin reductase protein with electron-transferring activity on the ferredoxin protein, 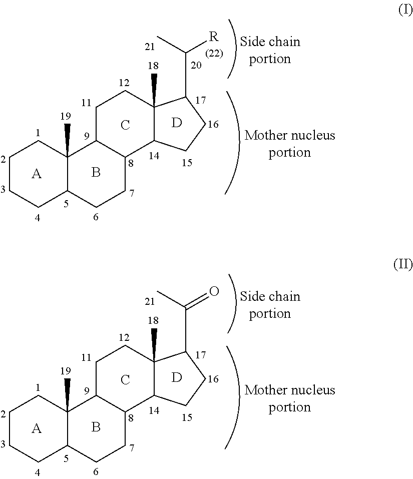
wherein the sterol is represented by the formula (I) of claim 2 and the compound is represented by the formula (II) of claim 2.

18. A method for producing a compound represented by formula (II), comprising contacting a sterol represented by formula (I) 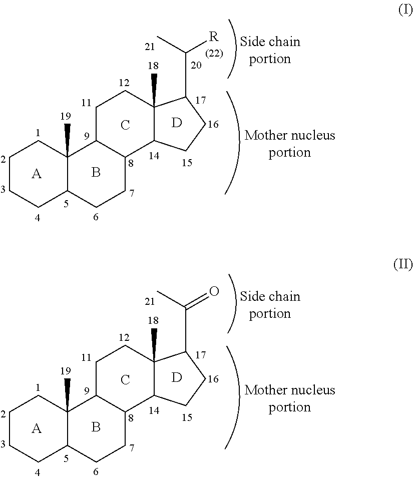
with the fusion protein of claim 5.

19. The method of claim 18, wherein the sterol is represented by the formula (I) of claim 2 and the compound is represented by the formula (II) of claim 2.

20. A method for producing a compound represented by formula (II), comprising contacting a sterol represented by formula (I) 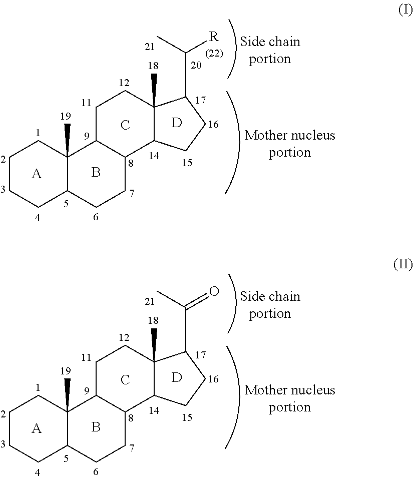
with a fusion protein, wherein
the protein of claim 2,
a ferredoxin protein having electron-transferring activity on the protein, and
a ferredoxin reductase protein having electron-transferring activity on the ferredoxin protein
are allowed to bind to one another, so that they can function as a single protein,
wherein the sterol is represented by the formula (I) of claim 2 and the compound is represented by the formula (II) of claim 2.

21. The method of claim 14, wherein the sterol is represented by formula (I) of claim 2.

22. A method for producing hydrocortisone, comprising culturing a yeast with a raw material in a medium containing the raw material,
wherein
the raw material is selected from the group consisting of glucose, glycerol, methanol, ethanol, sucrose, acetic acid and citric acid,
the yeast having an activity of generating a sterol represented by formula (I) of claim 2 from the raw material, 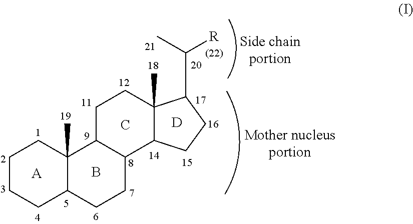
the yeast having an activity of generating at least one enzyme selected from the group consisting of 3beta-hydroxysteroid dehydrogenase, 3beta-hydroxysteroid:oxygen oxidoreductase, steroid 17α-hydroxylase, steroid 21-hydroxylase and steroid 11β-hydroxylase, and
the yeast having any one additional activity selected from the group consisting of:
(A) an activity (A) of generating a mixture of the protein of claim 2, a ferredoxin protein with electron-transferring activity on the protein, and a ferredoxin reductase protein having electron-transferring activity on the ferredoxin protein;
(B) an activity (B) of generating a mixture of a protein (B) consisting of the amino acid sequence shown in SEQ ID NO: 23, a ferredoxin protein (B) having electron-transferring activity on the protein (B), and a ferredoxin reductase protein (B) that transfers electrons to the ferredoxin protein (B);
(C) an activity (C) of generating a mixture of:
a protein (C) consisting of an amino acid sequence comprising deletion, substitution and/or addition of at least one amino acid with respect to the amino acid sequence shown in SEQ ID NO: 23, and having an activity (C1) of cleaving the bond between positions 20 and 22 of a sterol side chain, wherein the maximum velocity (Vmax) that is a reaction rate parameter of the activity (C1) is 40 mmol/min/mol or more;
a ferredoxin protein (C) having electron-transferring activity on the protein (C); and
a ferredoxin reductase protein (C) that transfers electrons to the ferredoxin protein (C);
(D) an activity (D) of generating a fusion protein (D), wherein
a protein (D) consisting of the amino acid sequence shown in SEQ ID NO: 23 or a protein consisting of an amino acid sequence comprising a deletion, substitution and/or addition of at least one amino acid with respect to the amino acid sequence shown in SEQ ID NO: 23, and having an activity (D) of cleaving the bond between positions 20 and 22 of a sterol side chain, wherein the maximum velocity (Vmax) that is a reaction rate parameter of the activity (D) is 40 mmol/min/mol or more,
a ferredoxin protein (D) having electron-transferring activity on the protein (D), and
a ferredoxin reductase protein (D) having electron-transferring activity on the ferredoxin protein (D)
are allowed to bind to one another, so that they can function as a single protein; and
(E) an activity (E) of generating a fusion protein (E), wherein
the protein of claim 2,
the a ferredoxin protein having electron-transferring activity on the protein, and
the a ferredoxin reductase protein having electron-transferring activity on the ferredoxin protein
are allowed to bind to one another, so that they can function as a single protein.
